(19) United States
(12) Patent Application Publication
(10) Pub. No.: US 2010/0261639 A1
(43) Pub. Date: Oct. 14, 2010

(54) TRIAZOLE-BASED AMINOGLYCOSIDE-PEPTIDE CONJUGATES AND METHODS OF USE

(75) Inventors: Frank Schweizer, Winnipeg (CA); George G. Zhanel, Winnipeg (CA); Smritilekha Bera, Troy, NY (US)

Correspondence Address: FULBRIGHT & JAWORSKI L.L.P., 600 CONGRESS AVE., SUITE 2400, AUSTIN, TX, 78701, US

(73) Assignee: UNIVERSITY OF MANITOBA, Winnipeg, MB (CA)

(21) Appl. No.: 12/602,318

(22) PCT Filed: May 28, 2008
(86) PCT No.: PCT/IB08/03486

§ 371 Date: Jun. 18, 2010

Related U.S. Application Data
(60) Provisional application No. 60/940,431, filed on May 28, 2007.

(51) Int. Cl. A61K 38/14; C07K 9/00; C07H 1/00; A61P 31/04

(52) U.S. Cl. 514/3.1; 530/322; 536/1.11; 514/20.9

(57) ABSTRACT

Aminoglycoside-amino acid and -peptide conjugates comprising a triazolyl linker are provided along with efficient methods of their preparation. The aminoglycoside may be an aminoglycoside antibiotic. Conjugates comprising an aminoglycoside antibiotic may exhibit antimicrobial activities against Gram-positive and/or Gram-negative strains and display significantly enhanced activity against multi-drug resistant MRSA and MRSE when compared to their unconjugated aminoglycoside antibiotic counterparts.

### CROSS REFERENCE TO RELATED APPLICATION

This application claims priority to U.S. Provisional Patent Application Ser. No. 60/940,431, filed May 28, 2007, which is incorporated by reference.

### BACKGROUND OF THE INVENTION

1. Field of the Invention

The present invention relates generally to the fields of click chemistry and antibacterial agents. More particularly, it concerns preparation of modified aminoglycosides as well as treatment of bacterial infections, such as multi-drug resistant bacterial infections, using triazole-linked aminoglycoside-amino acid and -peptide conjugates.

2. Description of the Related Art

Aminoglycoside antibiotics (AAs) constitute a large family of clinically important drugs used in the treatment of a variety of bacterial infections (Hooper, 1982; Haddad et al., 2001). Most of the naturally occurring AAs are structurally characterized by amino sugars glycosidically linked to an aminocyclitol which, in most cases, is 2-deoxystreptamine. Several types of 2-deoxy-streptamine derivatives exist: monosubstituted derivatives such as neamine, 4,5-disubstituted (neomycin type derivatives), and 4,6-disubstituted (kanamycin, tobramycin and gentamicin) derivatives. AAs carry up to six amino groups which are predominantly charged at physiological pH (Sitaram and Nagaraj, 2002; Gordon et al., 1994; Tenet et al., 1995; Bunin, 1998; Czarnik and De Witt, 1997) and bind with high affinity to anions and nucleic acids via electrostatic and hydrogen bonding interactions (Kolb and Sharpless, 2003; Kolb et al., 2001; Huisgen, 1961; Begg and Barclay, 1995). AAs are often characterized as broad-spectrum agents with activity against aerobic Gram-negative bacilli and certain Gram-positive cocci. These are bactericidal agents which bind to specific sites in prokaryotic ribosomal RNA (rRNA) affecting the fidelity of protein synthesis (Davis, 1987).

Since the discovery of streptomycin in 1944 (Waskman et al., 1944), several members of this class including amikacin, gentamicin, kanamycin, neomycin, netilmycin, streptomycin, and tobramycin have been used clinically for decades as potent antimicrobial agents (Vakulenko and Mobashery, 2003). Although AAs exhibit potent bactericidal activity, their worldwide use had decreased significantly due to well documented dose-related nephrotoxicity and ototoxicity (Kumar et al., 1980; Yoshikawa et al., 1984; Girodeau et al., 1984; Alper et al., 1998; Greenberg et al., 1999; Wang et al., 2002; Hanessian et al., 2003; Michael et al., 1999). Furthermore, as with other antibiotic regimens, their use as the primary treatment of life threatening infections has also been curtailed due to the global dissemination of aminoglycoside antibiotic resistant bacteria (Sucheck et al., 2000; Wang and Tor, 1993; Papagianni, 2003).

There are three general mechanisms of AA-resistance: (1) reduction of the intracellular concentration of the antibiotic within bacterial cells, usually via efflux of the agent out of the bacterial cell by either dedicated or general efflux pumps or other mechanisms (2) alteration of the molecular target of the antibiotic, usually as result of a spontaneous mutation in the gene encoding the target or substitution of the target's function by an exogenous gene; and (3) enzymatic inactivation of the aminoglycoside (Sucheck et al., 2000; Wang and Tor, 1993; Papagianni, 2003). The global emergence of AA-resistant strains has instigated research efforts to develop AA analogs that maintain activity against aminoglycoside antibiotic resistant strains as well as be able to delay or avoid acquired resistance by pathogenic bacteria. However, the development of synthetic aminoglycoside (AG) analogs faces several challenges. The polyfunctional nature of the AGs frequently requires multi-step organic synthesis involving many protection and deprotection steps. In addition, with the exception of neomycin and kanamycin, many commercially available AAs are expensive starting materials that limit their industrial use as synthetic scaffold for chemical modifications. Accordingly, avenues to conveniently produce novel AG analogs are needed.

### SUMMARY OF THE INVENTION

The present invention is based on the discovery and development of aminoglycoside (AG)-amino acid and -peptide conjugates comprising a triazoylyl moiety. Generally speaking, aminoglycoside-amino acid and -peptide conjugates of the present invention comprise at least one aminoglycoside, at least one amino acid, and at least one linker comprising triazolyl moiety to link at least one aminoglycoside to at least one amino acid. In certain embodiments, aminoglycoside-amino acid and -peptide conjugates of the present invention are further defended as triazole aminoglycoside-(amino acid)ₙ conjugates, wherein n is 1-20. In certain embodiments, triazole aminoglycoside-(amino acid)ₙ conjugates may induce synergistic effects due to dual warhead function: a polycationic pharmacophore of an aminoglycoside, such as an aminoglycoside antibiotic (AA), in the conjugate may enhance the electrostatic interactions with the lipid bilayer of bacteria, while a peptide moiety in the conjugate, such as a hydrophobic peptide moiety, may facilitate absorption and uptake or prevent efflux or covalent modification of the aminoglycoside.

Accordingly, the present invention contemplates a triazole aminoglycoside-(amino acid)ₙ conjugate, wherein at least one amino acid has been modified to comprise a triazolyl moiety, such as a triazolylmethyl linker, that is bound to at least one aminoglycoside, and n=1-20. The variable of “n” may also range higher than 20, such as up to 50 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or any range derivable therein (e.g., 1-50, 1-25, 1-20, 1-10, 1-5, 1-4, 1-3, or 1-2)). The amino acid may be modified in a variety of ways, as described herein. For example, a side chain of an amino acid may be modified to comprise a triazolylmethyl linker that is bound to at least one aminoglycoside. Alternatively or also, an N-terminus of an amino acid may be modified to comprise a triazolylmethyl linker that is bound to at least one aminoglycoside. Alternatively or also, a C-terminus of an amino acid may be modified to comprise a triazolylmethyl linker that is bound to at least one aminoglycoside.

An aminoglycoside may be bound to an amino acid in a variety of ways. In certain embodiments, the aminoglycoside is bound to a triazolylmethyl linker at a primary hydroxy position, a secondary hydroxy position, a primary amino position, or a secondary amino position of the aminoglycoside, as described herein. In certain embodiments, the aminoglycoside is bound to the triazolylmethyl linker at a primary hydroxy position of the aminoglycoside.

In any aspect of the present invention, an aminoglycoside may be further defined as an aminoglycoside antibiotic (AA). AAs are well-known in the art. In certain embodiments, an AA of the present invention is further defined as a neomycin, a kanamycin, amikacin, a gentamicin, neamine, a streptomycin, tobramycin, a hygromycin, or spectinomycin. In certain embodiments, the AA comprises a primary hydroxy position, such as in a neomycin, a kanamycin, amikacin, a streptomycin, tobramycin or a hygromycin. In particular embodiments, the AA is further defined as a neomycin or a kanamycin.

In certain embodiments regarding triazole aminoglycoside-(amino acid)ₙ conjugates of the present invention, n=1. In certain embodiments, n=2-20, each amino acid may be the same or different, and each amino acid is comprised in a single peptide. A single peptide may comprise a variety of amino acids or may comprise only one type of amino acid. In certain embodiments, the single peptide comprises one or more amino acid residues selected from the group consisting of L- or D-glycyl, L- or D-alanyl, L- or D-valinyl, L- or D-leucyl, L- or D-isoleucyl, L- or D-threonyl, L- or D-seryl, L- or D-cysteinyl, L- or D-methionyl, L- or D-aspartyl, L- or D-glutamyl, L- or D-histidyl, L- or D-lysinyl, L- or D-asparagyl, L- or D-glutaminyl, L- or D-arginyl, L- or D-phenylalanyl, L- or D-tyrosyl, L- or D-tryptophyl, or L- or D-prolinyl. In particular embodiments, the single peptide comprises L- or D-phenylalanyl, L- or D-tyrosyl, or L- or D-tryptophyl. In certain embodiments, the single peptide is further defined as a cationic antimicrobial peptide.

Propargyl groups may be introduced into amino acids and/or peptides, as described herein. Accordingly, in certain embodiments, the present invention contemplates a triazole aminoglycoside-(amino acid)ₙ conjugate, wherein n=1-50 (e.g., 1-20), and wherein at least one amino acid of the single peptide further comprises a propargyl group. For example, a side chain of the amino acid of the single peptide may be modified to comprise a propargyl group. Alternatively or also, an N-terminus of the amino acid of the single peptide may be modified to comprise a propargyl group. Alternatively or also, a C-terminus of an amino acid may be modified to comprise a propargyl group.

In certain embodiments, n=2 or 3 regarding a triazole aminoglycoside-(amino acid)ₙ conjugate. Non-limiting examples of such conjugates include:

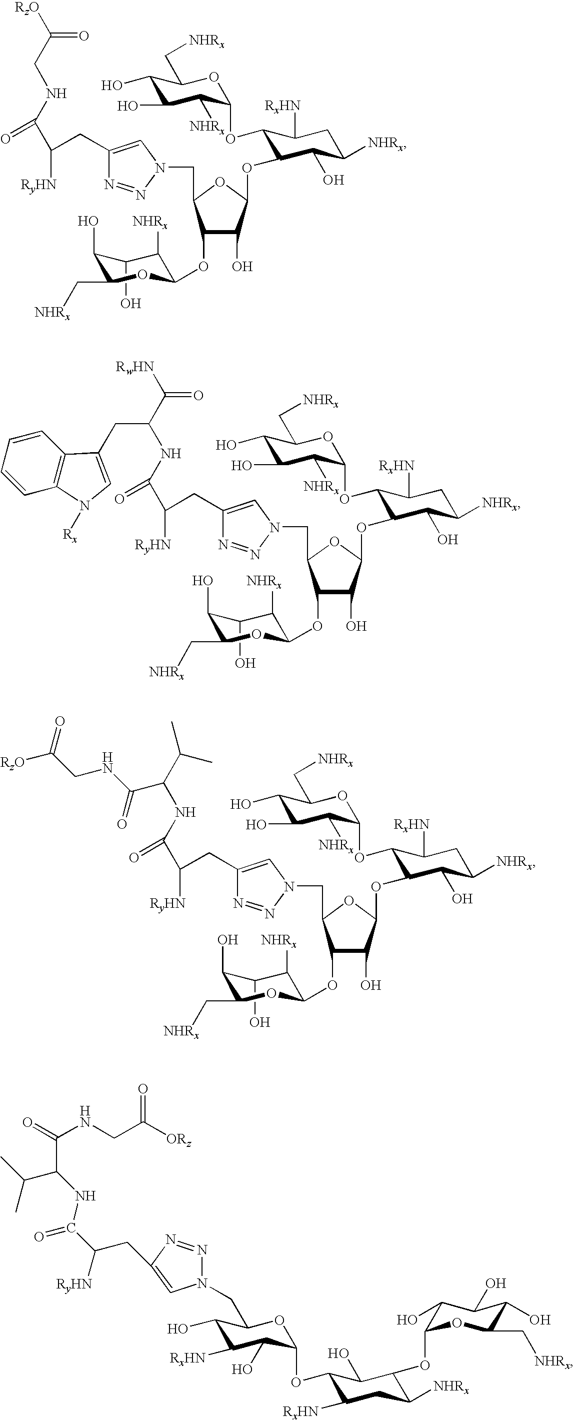

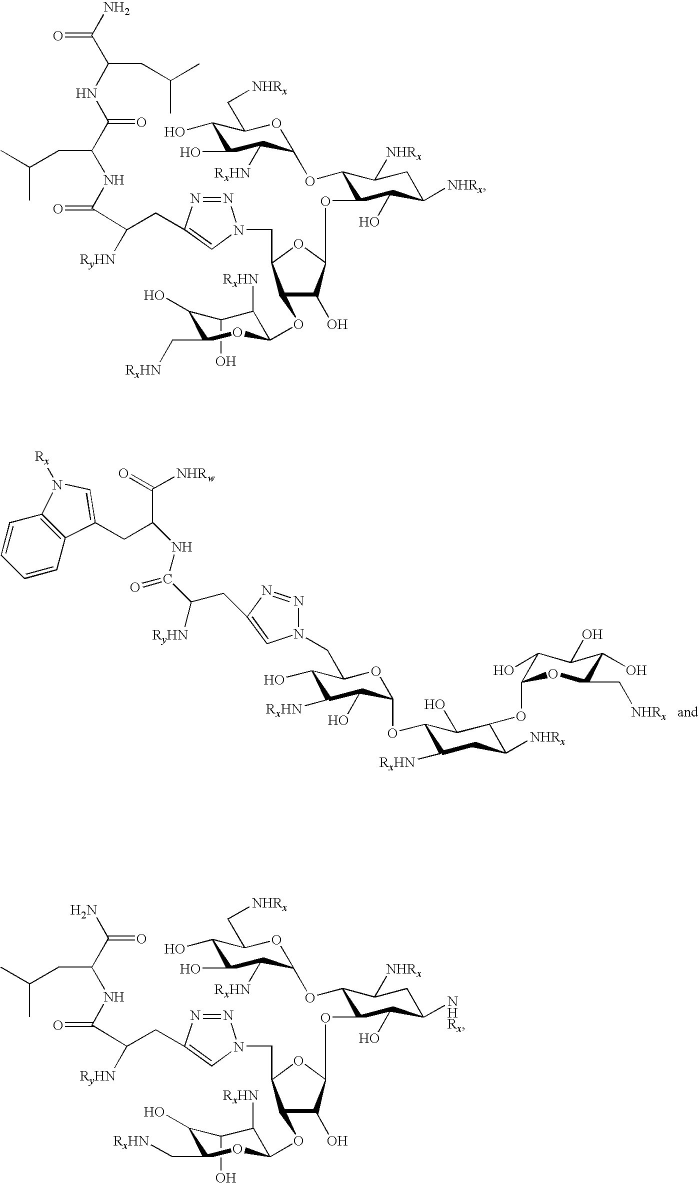

wherein: R_{w}, Rₓ and R_{y} are each independently H or an amine protecting group; and R_{z} is a carboxylic acid protecting group, or salts thereof.

In certain embodiments regarding triazole aminoglycoside-(amino acid)ₙ conjugates, at least two separate aminoglycosides are bound to at least two separate amino acids through two separate linkages that each comprise a triazolylmethyl linker. Non-limiting examples of such conjugates include:

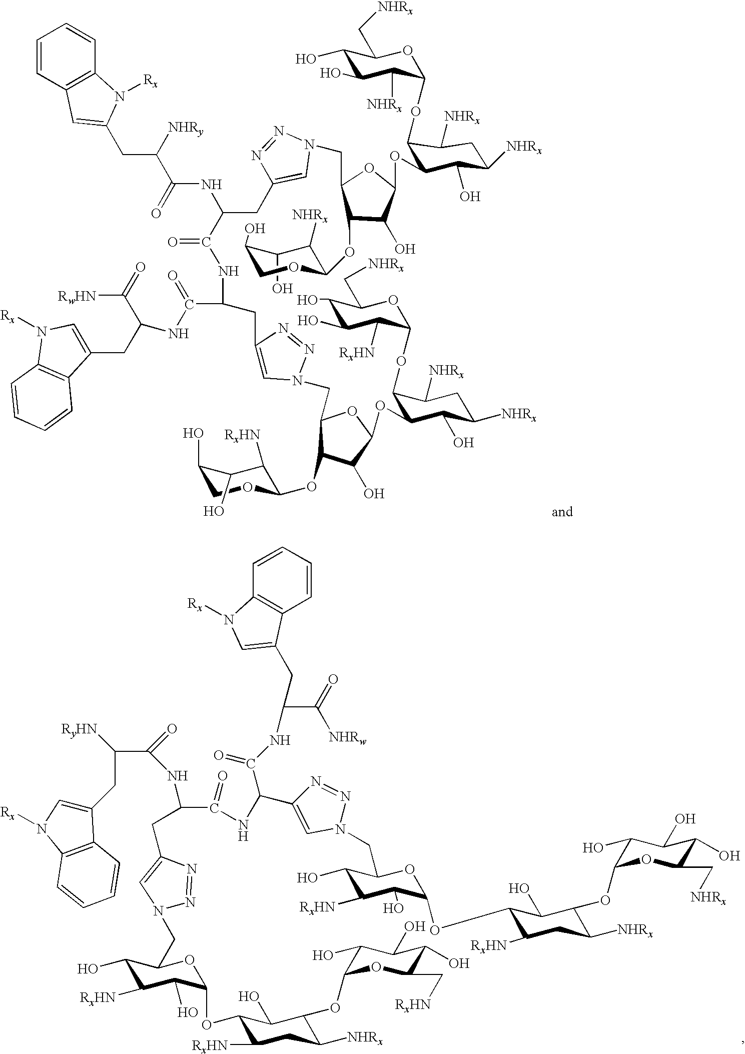

wherein: R_{w}, Rₓ and R_{y} are each independently H or an amine protecting group; and R_{z} is a carboxylic acid protecting group, or salts thereof.

In certain embodiments, a triazole aminoglycoside-(amino acid)ₙ conjugate of the present invention is defined as a compound of formula (I):

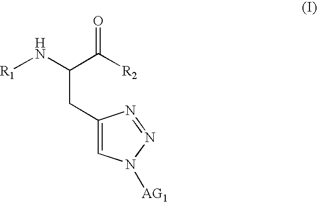

wherein: R₁ is H, an amino protecting group, or (aa₁)ᵣ, wherein (aa₁) is an amino acid that is bound to the —NH— group of the compound of formula (I) through its carboxyl terminus such that an amide bond is formed, and r=1-19; R₂ is —OR₃, wherein R₃ is H or a carboxylic acid protecting group, —NHR₄, wherein R₄ is H or an amino protecting group, or (aa₂)ₛ, wherein (aa₂) is an amino acid that is bound to the —C(O)— group of the compound of formula (I) such that an amide bond is formed, and s=1-19; and AG₁ is an aminoglycoside, wherein the triazolyl is bound to AG₁ at a primary hydroxy position of AG₁, wherein r+s≦20. In certain embodiments, R₁ is H or an amino protecting group. In certain embodiments, R₁ is (aa₁)ᵣ, and r=1. In certain embodiments, R₁ is (aa₁)ᵣ, and r=1-19. In certain embodiments, the amino acid in the terminal position of (aa₁)ᵣ terminates in —NHR₅, wherein R₅ is H or an amino protecting group. In certain embodiments, R₂ is —OR₃. In certain embodiments, R₂ is (aa₂)ₛ, and s=1. In certain embodiments, R₂ is (aa₂)ₛ, and s=1-19. In certain embodiments, the amino acid in the terminal position of (aa₂)ₛ terminates in —C(O)OR₆, wherein R₆ is —OH or a carboxylic acid protecting group, or —NHR₇, wherein R₇ is H or an amino protecting group. In certain embodiments, R₁ is (aa₁)ᵣ and R₂ is (aa₂)ₛ, and at least one amino acid of (aa₁)ᵣ or (aa₂)ₛ has been modified to comprise a triazolyl moiety, such as a triazolylmethyl linker, that is covalently bound to at least a second aminoglycoside (AG₂). The triazolyl moiety (e.g., triazolylmethyl linker) may be bound to the second AG₂ at a primary or secondary hydroxy or amino position of the AG₂. In certain embodiments, the triazolyl moiety (e.g., triazolylmethyl linker) is bound to the second AG₂ at a primary hydroxy position of the AG₂. In certain embodiments, R₁ is (aa₁)ᵣ and R₂ is (aa₂)ₛ, and at least one amino acid of of (aa₁)ᵣ or (aa₂)ₛ comprises a propargyl moiety.

Another general aspect of the present invention contemplates a peptide comprising the following moiety:

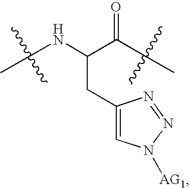

wherein AG₁ is an aminoglycoside that is bound to the triazolyl group at a primary or secondary hydroxy or amino position of AG₁. The AG₁ may be any aminoglycoside described herein, such as an aminoglycoside antibiotic. In certain embodiments, AG₁ is bound to the triazolyl moiety at the primary hydroxy position of AG₁.

Pharmaceutical compositions are also contemplated by the present invention, such as a pharmaceutical composition comprising a triazole aminoglycoside-(amino acid)ₙ conjugate, wherein the amino acid has been modified to comprise a triazolyl moiety (e.g., triazolylmethyl linker) that is bound to at least one aminoglycoside, and n=1-50 (e.g., 1-20), and a pharmaceutically acceptable carrier. The aminoglycoside may be any aminoglycoside described herein, such as an aminoglycoside antibiotic.

Methods of preparing triazole aminoglycoside-(amino acid)ₙ conjugates of the present invention are also contemplated by the present invention. Accordingly, one method of the present invention comprises a method of making a triazole aminoglycoside-(amino acid)ₙ conjugate wherein n=1-50 (e.g., 1-20), comprising reacting a first azido-modified aminoglycoside with a propargyl-modified amino acid. Such methods may further comprise the step of obtaining an azido-modified aminoglycoside, in certain embodiments. In certain embodiments, such methods may further comprise the step of obtaining a propargyl-modified amino acid. In certain embodiments, the azido-modified aminoglycoside is further defined as an aminoglycoside comprising a primary hydroxy position that has been modified to incorporate an azido group. Other positions may be modified as well, as described herein (e.g., a secondary hydroxy position, or a primary or secondary amino position). The propargyl-modified amino acid may be further defined as propargylglycine, for example. In certain embodiments, n=1-50 (e.g., 1-20), and each amino acid may be the same or different and each amino acid may be comprised in a single peptide. The single peptide may further comprise a second amino acid, wherein the second amino acid comprises a propargyl group. The propargyl group of the second amino acid may be further reacted, such as with a second azido-modified aminoglycoside, wherein the second aminoglycoside may be the same or different than the first aminoglycoside. In certain embodiments, the aminoglycoside-(amino acid)ₙ is further defined as an aminoglycoside antibiotic-(amino acid)ₙ and/or the azido-modified aminoglycoside is further defined as an azido-modified aminoglycoside antibiotic.

Methods of making triazole aminoglycoside-(amino acid)ₙ conjugates, such as those described above and below, may be performed using, for example, using solution phase peptide chemistry or solid phase peptide chemistry. Such techniques are well-known in the art.

Another general aspect of the present invention contemplates a method of making a compound of formula (I):

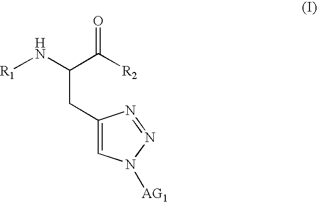

wherein: R₁ is H, an amino protecting group, or (aa₁)ᵣ, wherein (aa₁) is an amino acid that is bound to the —NH— group of the compound of formula (I) through its carboxyl terminus such that an amide bond is formed, and r=1-19; R₂ is —OR₃, wherein R₃ is H or a carboxylic acid protecting group, —NHR₄, wherein R₄ is H or an amino protecting group, or (aa₂)ₛ, wherein (aa₂) is an amino acid that is bound to the —C(O)— group of the compound of formula (I) such that an amide bond is formed, and s=1-19; and AG₁ is an aminoglycoside, wherein the triazolyl is bound to AG₁ at a primary hydroxy position of AG₁, wherein r=s≦20; comprising reacting an azido-modified-AG₁ with compound comprising a propargyl group, such as propargylglycine. The amino protecting groups may be orthogonal to, for example, facilitate synthesis. In certain embodiments, the compound comprising a propargy group is further defined as a peptide comprising a propargyl group, such as propargylglycine. Other propargyl-modified amino acids may be employed, as described herein.

Also contemplated by the present invention are methods of treating bacterial infections, such as a method of treating a bacterial infection in a subject comprising administering to the subject an effective amount of a triazole aminoglycoside antibiotic-(amino acid)ₙ conjugate, wherein at least one amino acid has been modified to comprise a triazolylmethyl linker that is bound to at least one aminoglycoside, and n=1-50 (e.g., 1-20). The bacterial infection may be caused by a variety of bacteria, such as a multi-drug resistant bacteria. The bacteria may be, for example, any of the following types: Staphylococcus aureus, MRSA, Staphylococcus epidermidis, MRSE, Enterococcus faecalis, Enterococcus faecium, Streptococcus pneumoniae, E. coli, Pseudomonas aeruginosa, Stenotrophomonas maltophilia, Acinetobacter baumannii, Klebsiella pneumoniae or Mycobacterium tuberculosis. In certain embodiments of such methods, the minimum inhibitory concentration of the triazole aminoglycoside antibiotic-(amino acid)ₙ conjugate (MIC) is ≦150 μg/mL. Such methods may also further comprise administration of a second antibacterial agent. Such methods may also further comprise diagnosing the subject as needing treatment for the bacterial infection prior to administering the triazole aminoglycoside antibiotic-(amino acid)ₙ conjugate. Such diagnoses are well-known in the art. A triazole aminoglycoside antibiotic-(amino acid)ₙ conjugate may be administered in a variety of ways to a subject, and in certain embodiments, a conjugate is topically administered to skin of the subject, wherein the skin has or is at risk of having a bacterial infection.

Methods of preventing bacterial infection comprising administering to a subject a triazole aminoglycoside-(amino acid)ₙ conjugate of the present invention to the subject are also contemplated. In such a method, a subject may be one at risk of bacterial infection: for example, such a subject may be about to enter an area known to contain bacteria that could cause an infection. The conjugate may be administered, for example, 1-3 days before the subject could be exposed to such bacteria, or 1-24 hours beforehand, or any range derivable therein.

As used herein, an “aminoglycoside” or “AG” refers a large and diverse class of antibiotics that characteristically contain two or more aminosugars linked by glycosidic bonds to an aminocyclitol component. Examples of aminoglycosides are neomycin, kanamycin, tobramycin, neamine, streptomycin and others. An “aminoglycoside antibiotic” or “AA” refers to a class of aminoglycosides that exhibit concentration-dependent antibacterial activity. See, e.g., Hooper, 1982; Haddad et al., 2001.

As used herein, “a primary hydroxy position of the aminoglycoside” refers to an aminoglycoside that comprises a primary hydroxy group, such that that position is the “primary hydroxy position.” The phrases regarding “a secondary hydroxy group of an aminoglycoside,” “a primary amino group of an aminoglycoside” and “a secondary amino group of an aminoglycoside” may be interpreted similarly. For example, kanamycin A, as that compound is known in the art, contains only one primary hydroxy group, only one primary amino group, and several secondary hydroxy and amino groups. Accordingly, kanamyin A contains only one primary hydroxy position, only one primary amino position, and contains several secondary hydroxy and amino positions. As another example, neomycin B contains only one primary hydroxy group, only two primary amino groups, and several secondary hydroxy and amino groups. Accordingly, neomycin B contains only one primary hydroxy position, only two primary amino positions, and several secondary hydroxy and amino positions. Moreover, an aminoglycoside antibiotic may be modified to contain a primary or secondary hydroxy or amino group. When a moiety is bound to an aminoglycoside at, e.g., “a primary hydroxy position of the aminoglycoside,” it means that the primary hydroxy group has been modified such that the moiety is now bound at that primary hydroxy position. The same reasoning may be applied to moieties bound to secondary hydroxy positions, primary amino positions, and secondary amino positions.

As used herein, “cationic antimicrobial peptides” (AMPs) are characterized by a net excess of positively charged residues, the presence of hydrophobic residues (side chains of natural and unnatural aromatic amino acids including tryptophan, phenylalanine and tyrosine but also chains of lipophilic amino acids such as valine, leucine, isoleucine and others) and a typical size ranging from 12 to 50 residues (Vakulenko and Mobashery, 2003).

As used herein, a “multidrug resistant (MDR) bacteria” is resistant to two or more antimicrobial classes. For example, MDRTB (tuberculosis) is used to describe strains that are resistant to two or more of the five first-line anti-TB drugs (isoniazid, rifampin, pyrizinamide, ethambutol and streptomycin).

As used herein, an “amino acid” refers to any of the naturally occurring amino acids, as well as synthetic analogs (e.g., D-stereoisomers of the naturally occurring amino acids, such as D-threonine). α-Amino acids comprise a carbon atom to which is bonded an amino group, a carboxyl group, a hydrogen atom, and a distinctive group referred to as a “side chain.” β- and γ-Amino acids are also known in the art and are contemplated by the present invention. The side chains of naturally occurring amino acids are well known in the art and include, for example, hydrogen (e.g., as in glycine), alkyl (e.g., as in alanine, valine, leucine, isoleucine, proline), substituted alkyl (e.g., as in threonine, serine, methionine, cysteine, aspartic acid, asparagine, glutamic acid, glutamine, arginine, and lysine), arylalkyl (e.g., as in phenylalanine and tryptophan), substituted arylalkyl (e.g., as in tyrosine), and heteroarylalkyl (e.g., as in histidine). Unnatural amino acids are also known in the art, as set forth in, for example, Williams (1989); Evans et al. (1990); Pu et al. (1991); Williams et at (1991); and all references cited therein. The present invention thus includes unnatural amino acids and their side chains as well. Further, an amino acid may comprise a propargyl group in its side chain (e.g., propargylglycine) (Pra). Protected amino acids are also contemplated, such as when an N-terminus, C-terminus, and/or functional group of a side chain is protected by a protecting group.

Moreover, an “amino acid” refers to both an amino acid, alone (e.g., glycine), or an amino acid residue (e.g., glycyl). When two or more amino acids combine to form a peptide and the elements of water are removed, what remains of each amino acid may be called an “amino acid residue.” Amino-acid residues are structures that lack a hydrogen atom of the amino group (—NH—CHR—COOH), or the hydroxyl moiety of the carboxyl group (NH₂—CHR—CO—), or both (—NH—CHR—CO—); all units of a peptide chain are therefore amino acid residues. Amino acids may terminate in —COOH, —COO(R), wherein R is a carboxylic acid protecting group, —C(O)NHR₁, or —NHR₂, wherein R₁ and R₂ are each independently H or an amino protecting group.

As used herein, a “peptide” refers to two or more amino acids joined together by an amide bond. Peptides may terminate in any fashion described above regarding amino acids. “Ultrashort” peptides refer to di-, tri- and tetra-peptides. In certain embodiments, peptides comprise up to or include 50 amino acids.

As used herein, the word “link,” “linkage,” “linker,” or “bound” refers to covalent binding between species, unless specifically noted otherwise.

As used herein, “protecting group” refers to a moiety attached to a functional group to prevent an otherwise unwanted reaction of that functional group. The term “functional group” generally refers to how persons of skill in the art classify chemically reactive groups. Examples of functional groups include hydroxyl, amine, sulfhydryl, amide, carboxyl, carbonyl, etc. Protecting groups are well-known to those of skill in the art. Non-limiting exemplary protecting groups fall into categories such as hydroxy protecting groups, amino protecting groups, sulfhydryl protecting groups and carbonyl protecting groups. Such protecting groups, including examples of their installation and removal, may be found in Greene and Wuts, 1999, incorporated herein by reference in its entirety. Triazole aminoglycoside-(amino acid)ₙ conjugates described herein are contemplated as protected by one or more protecting groups—that is, the present invention contemplates such conjugates in their “protected form.” Non-limiting examples of carboxylic acid protecting groups include benzyl (Bn) and t-butyl. Non-limiting examples of amino protecting groups include Bn, carbobenzyloxy (Cbz), t-butoxycarbonyl (Boc) and 9-fluorenylmethyloxycarbonyl (Fmoc), for example.

Compounds of the present invention may contain one or more asymmetric centers and thus can occur as racemates and racemic mixtures, single enantiomers, diastereomeric mixtures and individual diastereomers. In certain embodiments, a single diastereomer is present. All possible stereoisomers of the compounds of the present invention are contemplated as being within the scope of the present invention. However, in certain aspects, particular diastereomers are contemplated. The chiral centers of the compounds of the present invention can have the S- or the R-configuration, as defined by the IUPAC 1974 Recommendations. In certain aspects, certain compounds of the present invention may comprise S- or R-configurations at particular carbon centers.

Synthetic techniques that may be used to prepare certain compounds of the present invention are provided in the Examples section. Other synthetic techniques to prepare compounds of the present invention as well as derivatives are well-known to those of skill in the art. For example, Smith and March, 2001 discuss a wide variety of synthetic transformations, reaction conditions, and possible pitfalls relating thereto, including amidation and esterification reactions. Methods of solution and solid phase peptide chemistry are also well known. See, e.g., Bodansky, 1993 and Grant, 1992, each of which is incorporated herein by reference. Methods discussed therein may be adapted to prepare compounds of the present invention from commerically available starting materials.

Solvent choices for preparing compounds of the present invention will be known to one of ordinary skill in the art. Solvent choices may depend, for example, on which one(s) will facilitate the solubilizing of all the reagents or, for example, which one(s) will best facilitate the desired reaction (particularly when the mechanism of the reaction is known). Solvents may include, for example, polar solvents and non-polar solvents. Solvents choices include, but are not limited to, tetrahydrofuran, dimethylformamide, dimethylsulfoxide, dioxane, methanol, ethanol, hexane, methylene chloride and acetonitrile. More than one solvent may be chosen for any particular reaction or purification procedure. Water may also be admixed into any solvent choice. Further, water, such as distilled water, may constitute the reaction medium instead of a solvent.

Persons of ordinary skill in the art will be familiar with methods of purifying compounds of the present invention. One of ordinary skill in the art will understand that compounds of the present invention can generally be purified at any step, including the purification of intermediates as well as purification of the final products. In certain embodiments, purification is performed via silica gel column chromatography or HPLC.

Modifications or derivatives of the compounds, agents, and active ingredients disclosed throughout this specification are contemplated as being useful with the methods and compositions of the present invention. Derivatives may be prepared and the properties of such derivatives may be assayed for their desired properties by any method known to those of skill in the art, such as methods described herein.

In certain aspects, “derivative” refers to a chemically-modified compound that still retains the desired effects of the compound prior to the chemical modification. A “triazole aminoglycoside-(amino acid)ₙ conjugate derivative,” therefore, refers to a chemically modified triazole aminoglycoside-(amino acid)ₙ conjugate that still retains the desired effects of the parent triazole aminoglycoside-(amino acid)ₙ conjugate prior to its chemical modification. Such effects may be enhanced (e.g., slightly more effective, twice as effective, etc.) or diminished (e.g., slightly less effective, 2-fold less effective, etc.) relative to the parent triazole aminoglycoside-(amino acid)ₙ conjugate, but may still be considered a triazole aminoglycoside-(amino acid)ₙ conjugate derivative. Such derivatives may have the addition, removal, or substitution of one or more chemical moieties on the parent molecule. Non-limiting examples of the types of modifications that can be made to the compounds and structures disclosed herein include the addition or removal of lower unsubstituted alkyls such as methyl, ethyl, propyl, or substituted lower alkyls such as hydroxymethyl or aminomethyl groups; carboxyl groups and carbonyl groups; hydroxyls; nitro, amino, amide, imide, and azo groups; sulfate, sulfonate, sulfono, sulfhydryl, sulfenyl, sulfonyl, sulfoxido, sulfonamide, phosphate, phosphono, phosphoryl groups, and halide substituents. Additional modifications can include an addition or a deletion of one or more atoms of the atomic framework, for example, substitution of an ethyl by a propyl, or substitution of a phenyl by a larger or smaller aromatic group. Alternatively, in a cyclic or bicyclic structure, heteroatoms such as N, S, or O can be substituted into the structure instead of a carbon atom.

As used herein, the term “patient” or “subject” refers to a living mammalian organism, such as a human, monkey, cow, sheep, goat, dogs, cat, mouse, rat, guinea pig, or transgenic species thereof. In certain embodiments, the patient or subject is a primate. Non-limiting examples of human subjects are adults, juveniles, infants and fetuses.

The claimed invention is also intended to encompass salts of any of the compounds of the present invention. The term “salt(s)” as used herein, is understood as being acidic and/or basic salts formed with inorganic and/or organic acids and bases. Zwitterions (internal or inner salts) are understood as being included within the term “salt(s)” as used herein, as are quaternary ammonium salts such as alkylammonium salts. Nontoxic, pharmaceutically acceptable salts are preferred, although other salts may be useful, as for example in isolation or purification steps during synthesis. Salts include, but are not limited to, sodium, lithium, potassium, amines, tartrates, citrates, hydrohalides, phosphates and the like.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means salts of compounds of the present invention which are pharmaceutically acceptable, as defined above, and which possess the desired pharmacological activity. Compounds of the present invention are contemplated in their pharmaceutically acceptable salt forms. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylicacids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, Selection and Use (P. H. Stahl & C. G. Wermuth eds., Verlag Helvetica Chimica Acta, 2002), which is incorporated herein by reference.

“Prodrug” means a compound that is convertible in vivo metabolically into a triazole aminoglycoside-(amino acid)ₙ conjugate, according to the present invention. Such prodrugs are contemplated by the present invention. The prodrug itself may or may not also have activity with respect to a given target. For example, a compound comprising a hydroxy group may be administered as an ester that is converted by hydrolysis in vivo to the hydroxy compound. Suitable esters that may be converted in vivo into hydroxy compounds include acetates, citrates, lactates, phosphates, tartrates, malonates, oxalates, salicylates, propionates, succinates, fumarates, maleates, methylene-bis-b-hydroxynaphthoates, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Similarly, a compound comprising an amine group may be administered as an amide that is converted by hydrolysis in vivo to the amine compound.

Hydrates of compounds of the present invention are also contemplated. The term “hydrate” when used as a modifier to a compound means that the compound has less than one (e.g., hemihydrate), one (e.g., monohydrate), or more than one (e.g., dehydrate) water molecules associated with each compound molecule, such as in solid forms of the compound.

The terms “inhibiting” or “reducing” or any variation of these terms, when used in the claims and/or the specification, includes any measurable decrease or complete inhibition to achieve a desired result. For example, there may be a decrease of 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of bacterial infection following administration of a triazole aminoglycoside-(amino acid)ₙ conjugate of the present invention. In a further example, following administering of a triazole aminoglycoside-(amino acid)ₙ conjugate of the present invention, a patient suffering from a bacterial infection may experience a reduction the number and/or intensity of symptoms of the infection. Non-limiting examples of typical symptoms associated with a bacterial infection include elevated temperature, sweating, chills, and/or excess white blood cells compared to a normal range.

The term “effective,” as that term is used in the specification and/or claims, means adequate to accomplish a desired, expected, or intended result.

“Therapeutically effective amount” means that amount which, when administered to an animal for treating a disease, condition, or infection, is sufficient to effect such treatment for the disease, condition, or infection.

“Treatment” or “treating” includes (1) inhibiting a disease, condition, or infection in a subject or patient experiencing or displaying the pathology or symptomatology of the disease, condition, or infection (e.g., arresting further development of the pathology and/or symptomatology), (2) ameliorating a disease, condition, or infection in a subject or patient that is experiencing or displaying the pathology or symptomatology of the disease, condition, or infection (e.g., reversing the pathology and/or symptomatology), and/or (3) effecting any measurable decrease in a disease, condition, or infection in a subject or patient that is experiencing or displaying the pathology or symptomatology of the disease, condition, or infection.

“Prevention” or “preventing” includes: (1) inhibiting the onset of a disease, condition, or infection in a subject or patient which may be at risk and/or predisposed to the disease, condition, or infection but does not yet experience or display any or all of the pathology or symptomatology of the disease, condition, or infection, and/or (2) slowing the onset of the pathology or symptomatology of a disease, condition, or infection in a subject of patient which may be at risk and/or predisposed to the disease, condition, or infection but does not yet experience or display any or all of the pathology or symptomatology of the disease, condition, or infection.

As used herein the specification, “a” or “an” may mean one or more, unless clearly indicated otherwise. As used herein in the claim(s), when used in conjunction with the word “comprising,” the words “a” or “an” may mean one or more than one. As used herein “another” may mean at least a second or more.

The use of the term “or” in the claims is used to mean “and/or” unless explicitly indicated to refer to alternatives only or the alternative are mutually exclusive, although the disclosure supports a definition that refers to only alternatives and “and/or.”

Throughout this application, the term “about” is used to indicate that a value includes the inherent variation of error for the device, the method being employed to determine the value, or the variation that exists among the study subjects.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein.

FIG. 1 Neomycin B- and kanamycin A-derived monoazido aminoglycosides used in glycoconjugation to peptides. The azide substituent in each is positioned at the C5″ position in neomycin B and at the C6″ position in kanamycin A.

FIG. 2 Selected hydrophobic di-, tri- and tetrapeptides for glycoconjugation with aminoglycoside-based azides 1 and 2.

FIG. 3 Synthesis of peptides 3-6.

FIG. 4. Glycoconjugation of neomycin B-derived azide 1 with peptide 3.

FIG. 5 Click-based glycoconjugation of peptides 4 and 5 with neomycin-based azide 1.

FIG. 6 Click-based glycoconjugation of peptides 10 and 11 with kanamycin A-based azide 2.

FIG. 7. Synthesis of bisaminoglycoside-peptide conjugates 20-23.

FIG. 8 Synthesis of aminoglycoside-peptide conjugates 24 and 25 on the solid-phase.

### DESCRIPTION OF THE ILLUSTRATIVE EMBODIMENTS

### I. Click Chemistry

### A. Background

“Click chemistry” is a chemical philosophy introduced by K. Barry Sharpless in 2001 and describes chemistry tailored to generate substances quickly and reliably by joining small units together. See, e.g., world wide web at sigmaaldrich.com/Area_of_Interest/Chemistry/Chemical_Synthesis/Product_Highligh ts/Click.html. The term “click chemistry” is often applied to a collection of supremely reliable and self-directed organic reactions (Kolb et al., 2001). For example, the identification of the copper catalyzed azide-alkyne [3+2] cycloaddition as a highly reliable molecular connection in water (Rostovtsev et al., 2002) has been used to augment several types of investigations of biomolecular interactions (Wang et al., 2003; Speers et al., 2003; Link and Tirrell, 2003; Deiters et al., 2003). In addition, applications to organic synthesis (Lee et al., 2003), drug discovery (Kolb and Sharpless, 2003; Lewis et al., 2002) and the functionalization of surfaces (Meng et al., 2004; Fazio et al., 2002) have also appeared.

The copper-catalyzed azide-alkyne ligation process has emerged as a unique combination of selective reactivity and “bullet-proof” scope (Rostovtsev et al., 2002; Tornøe et al., 2002). The use of Cu(I) catalysts may accelerate a reaction by factors up to 107 while preserving the inertness of both azides and alkynes towards a vast majority of functional groups and conditions (Rostovtsev et al., 2002; Wang et al., 2003).

### B. The Present Invention

One non-limiting method of making compounds of the present invention involves click chemistry. For example, a propargyl group may be incorporated into an amino acid or peptide, and then the propargyl-modified amino acid or propargyl-modified peptide may be conjugated to an azido-modified aminoglycoside, such as an azido-modified aminoglycoside antibiotic, using click chemistry as described herein.

Amino acids and peptides may be modified to comprise a propargyl group in a variety of ways. For example, a side chain of an amino acid (singly or as comprised in a peptide) may be modified to comprise a propargyl group. Propargylglycine may be prepared, for example, or purchased. Serine, threonine and other amino acids that comprise a side chain hydroxy group may be modified with a propargyl group, such as through esterification using propargylic acid. Esterification methods are well-known in the art. See, e.g., Smith and March, 2001, incorporated herein by reference. Propargylic acid may be used to modify side chains containing amino functional groups, such as found in lysine, ornithine and diaminobutyric acid, via amidation reactions. Amidation reactions are also well-known in the art, and various methods are discussed in Smith and March, 2001. Furthermore, the N-terminus of an amino acid or a peptide may be modified to comprise a propargyl group, such as through amidation in the presence of propargylic acid. Moreover, the C-terminus of an amino acid or a peptide may be modified to comprise a propargyl group, such as through esterification in the presence of propargylic acid. An amino acid or peptide may be modified in more than one way to comprise more than one propargyl group.

Aminoglycosides may be modified to comprise an azido group in a variety of ways. An azido group may be introduced at a primary hydroxy position, a secondary hydroxy position, a primary amido position, or a secondary amido position of an aminoglycoside (or any combination thereof). See, e.g., Disney et al., 2007 and Quader et al., 2007, each of which is incorporated herein by reference.

### II. Cationic Antimicrobial Peptides

Certain triazole aminoglycoside-(amino acid)ₙ conjugate s of the present invention comprise a cationic antimicrobial peptide. Cationic antimicrobial peptides (AMPs) form a diverse class of antibiotics and are characterized by a net excess of positively charged residues, the presence of hydrophobic residues and a typical size ranging from 12 to 50 residues (Vakulenko and Mobashery, 2003). Although the mode of action of AMPs is not fully understood, most AMPs appear to manifest their biological action by enhancing the permeability of lipid membranes of bacterial cells. This typically involves initial electrostatic interactions between the positively charged basic side chains to the negatively charged lipid membrane of pathogens, followed by adoption of an amphipathic α-helical or β-sheet structure (Vakulenko and Mobashery, 2003; Mingeot-Leclercq and Tulkens, 1999). The ability to kill target bacteria rapidly, an unusually broad spectrum of activity against some of the more serious antibiotic resistant pathogens and relative difficulty with which mutants develop resistance in vitro make AMPs attractive targets for drug development (Vakulenko and Mobashery, 2003; Mingeot-Leclercq and Tulkens, 1999). However, in vivo efficacy studies of several cationic peptide antibiotics have been disappointing most likely due to the fact that many AMPs exhibit poor bioavailability, susceptibility to proteolytic cleavage and low in vivo antimicrobial activity (Magnet and Blanchard, 2005; Fong and Berghuis, 2002; Smith and baker, 2002; Davies, 1994; Shaw et al., 1993). Moreover, the size of most AMPs is so large that production costs represent additional concern.

In order to overcome some of these drawbacks, ultrashort cationic antimicrobial peptides in the form of di-, tri- and tetrapeptides have recently emerged as a novel class of potential antimicrobial drug candidates (Asensio et al., 2005; Hanessian et al., 1977; Bastida et al., 2006). In particular, the small size and facile preparation reduce production costs while the presence of only a few amide bonds and the low molecular weight may improve the pharmacokinetic and pharmacodynamic properties of ultrashort cationic antimicrobial peptides.

As noted above, triazole aminoglycoside-(amino acid)ₙ conjugates of the present invention may, in certain embodiments, induce synergistic antibiotic effects due to dual warhead functionalities associated with the AG or AA and the peptide moieties.

### III. Pharmaceutical Formulations and Administration

Pharmaceutical compositions of the present invention comprise an effective amount of one or more candidate substances (e.g., a triazole aminoglycoside-(amino acid)ₙ conjugate) or additional agents dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one candidate substance or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, pp 1289-1329, 1990). Except insofar as any conventional carrier is incompatible with the active ingredient, its use in the therapeutic or pharmaceutical compositions is contemplated.

The candidate substance may comprise different types of carriers depending on whether it is to be administered in solid, liquid or aerosol form, and whether it needs to be sterile for such routes of administration as injection. Compounds of the present invention may be administered orally, intraadiposally, intraarterially, intraarticularly, intracranially, intradermally, intralesionally, intramuscularly, intranasally, intraocularally, intrapericardially, intraperitoneally, intrapleurally, intraprostaticaly, intrarectally, intrathecally, intratracheally, intraumbilically, intravaginally, intravenously, intravesicularlly, intravitreally, liposomally, locally, mucosally, orally, parenterally, rectally, subconjunctival, subcutaneously, sublingually, topically, transbuccally, transdermally, vaginally, in crèmes, in lipid compositions, via a catheter, via a lavage, via continuous infusion, via infusion, via inhalation, via injection, via local delivery, via localized perfusion, bathing target cells directly, or by other method or any combination of the foregoing as would be known to one of ordinary skill in the art (see, for example, Remington's Pharmaceutical Sciences, 1990). In particular embodiments, the composition may be formulated for oral delivery. In certain embodiments, intramuscular, intravenous, topical administration, or inhalation administration is contemplated. Pharmaceutical compositions comprising a compound of the present invention are also contemplated, and such compositions may be adapted for administration via any method known to those of skill in the art, such as the methods described above.

In particular embodiments, the composition is administered to a subject using a drug delivery device. Any drug delivery device is contemplated for use in delivering a pharmaceutically effective amount of a triazole aminoglycoside-(amino acid)ₙ conjugate.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will typically determine the concentration of active ingredient(s) in a composition and appropriate dose(s) for the individual subject. Compounds of the present invention may, in certain embodiments, be cleared by the kidneys: thus, it may, in certain embodiments, be important to assess any underlying problems with kidney function. Kidney function may be assessed by measuring the blood levels of creatinine, a protein normally found in the body. If these levels are higher than normal, it is an indication that the kidneys may not be functioning at an optimal rate and dosage may be lowered accordingly

The dose can be repeated as needed as determined by those of ordinary skill in the art. Thus, in some embodiments of the methods set forth herein, a single dose is contemplated. In other embodiments, two or more doses are contemplated. Where more than one dose is administered to a subject, the time interval between doses can be any time interval as determined by those of ordinary skill in the art. For example, the time interval between doses may be about 1 hour to about 2 hours, about 2 hours to about 6 hours, about 6 hours to about 10 hours, about 10 hours to about 24 hours, about 1 day to about 2 days, about 1 week to about 2 weeks, or longer, or any time interval derivable within any of these recited ranges.

In certain embodiments, it may be desirable to provide a continuous supply of a pharmaceutical composition to the patient. This could be accomplished by catheterization, followed by continuous administration of the therapeutic agent, for example. The administration could be intra-operative or post-operative.

In certain embodiments, pharmaceutical compositions may comprise, for example, at least about 0.1% of a triazole aminoglycoside-(amino acid)ₙ conjugate. In other embodiments, the triazole aminoglycoside-(amino acid)ₙ conjugatemay comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal, or combinations thereof.

The triazole aminoglycoside-(amino acid)ₙ conjugatemay be formulated into a composition, such as a pharmaceutical composition, in a free base, neutral, or salt form. Pharmaceutically acceptable salts are described herein.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. It may be preferable to include isotonic agents, such as, for example, sugars, sodium chloride, or combinations thereof.

In other embodiments, one may use eye drops, nasal solutions or sprays, aerosols or inhalants in the present invention. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in certain embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments the candidate substance is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. In certain embodiments, carriers for oral administration comprise inert diluents (e.g., glucose, lactose, or mannitol), assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain embodiments an oral composition may comprise one or more binders, excipients, disintegration agents, lubricants, flavoring agents, or combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar, or both.

Sterile injectable solutions may be prepared by incorporating a compound of the present invention in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, certain methods of preparation may include vacuum-drying or freeze-drying techniques which yield a powder of the active ingredient plus any additional desired ingredient from a previously sterile-filtered liquid medium thereof. The liquid medium should be suitably buffered if necessary and the liquid diluent (e.g., water) first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition should be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin, or combinations thereof.

### IV. Combination Therapy

In order to enhance or increase the effectiveness of a triazole aminoglycoside-(amino acid)ₙ conjugate of the present invention, the conjugate may be combined with another therapy, such as another agent that combats and/or prevents bacterial infection. For example, triazole aminoglycoside-(amino acid)ₙ conjugates of the present invention may be provided in a combined amount with an effective amount of an anti-bacterial agent (that is, an antibiotic). Anti-bacterial classes and agents are well-known in the art, and include, for example, the classes of aminoglycoside antibiotics, cephalosporins, penicillins, quinolones, sulfonamides, tetracyclines, beta-lactams and macrolides. Non-limiting specific examples of antibacterial agents include linezolid, tigecycline, tetracycline, oxytetracycline, doxycycline, minocycline, vancomycin, enrofloxacin, erythromycin, tyrocidine, griseofulvin, streptomycin, polymyxin, cephalosporin, ampicillin, cephalothin, lincomycin, gentamicin, carbenicillin, cephalexin and clindamycin. These lists of antibiotics are not exhaustive and one skilled in the art can readily determine other antibiotics which may be employed.

It is contemplated that combination therapy of the present invention may be used in vitro or in vivo. These processes may involve administering the agents at the same time or within a period of time wherein separate administration of the substances produces a desired therapeutic benefit. This may be achieved by contacting the cell, tissue, or organism with a single composition or pharmacological formulation that includes two or more agents, or by contacting the cell with two or more distinct compositions or formulations, wherein one composition includes one agent and the other includes another.

The compounds of the present invention may precede, be co-current with and/or follow the other agents by intervals ranging from minutes to weeks. In embodiments where the agents are applied separately to a cell, tissue or organism, one would generally ensure that a significant period of time did not expire between the time of each delivery, such that the agents would still be able to exert an advantageously combined effect on the cell, tissue or organism. For example, in such instances, it is contemplated that one may contact the cell, tissue or organism with two, three, four or more modalities substantially simultaneously (i.e., within less than about a minute) as the candidate substance. In other aspects, one or more agents may be administered about 1 minute, about 5 minutes, about 10 minutes, about 20 minutes about 30 minutes, about 45 minutes, about 60 minutes, about 2 hours, about 3 hours, about 4 hours, about 5 hours, about 6 hours, about 7 hours about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 1, about 2, about 3, about 4, about 5, about 6, about 7 or about 8 weeks or more, and any range derivable therein, prior to and/or after administering the candidate substance.

Various combination regimens of the agents may be employed. Non-limiting examples of such combinations are shown below, wherein a triazole aminoglycoside-(amino acid)ₙ conjugate is “A” and a second agent, such as an anti-bacterial agent, is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

### V. Examples

The following examples are included to demonstrate particular embodiments of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

### Example 1

### Materials and Methods for Examples 2-7

NMR spectra were recorded on a Brucker Avance 300 spectrometer (300 MHz for ¹H NMR, 75 MHz for ¹³C) and AMX 500 spectrometer (500 MHz for ¹H NMR). Optical rotation was measured at a concentration of g/100 mL, with a Perkin-Elmer polarimeter (accuracy)(0.002°. GC-MS analyses were performed on a Perkin-Elmer Turbomass-Autosystem XL. Analytical thin-layer chromatography was performed on precoated silica gel plates. Visualization was performed by ultraviolet light and/or by staining with ninhydrine solution in ethanol. Chromatographic separations were performed on a silica gel column by flash chromatography (Kiesel gel 40, 0.040-0.063 mm; Merck). Yields are given after purification, unless differently stated. When reactions were performed under anhydrous conditions, the mixtures were maintained under nitrogen. Compounds were named following IUPAC rules as applied by Beilstein-Institute AutoNom (version 2.1) software for systematic names in organic chemistry.

### Example 2

### Preparation of Neomycin and Kanamycin A Trizole Conjugates

Neomycin B and kanamycin A were initially selected for modification. As a method of ligation, the inventors selected the Sharpless modified Huisgen [3+2] cycloaddition reaction between a terminal peptide-based alkyne introduced in the form of propargylglycine (Pra) and an aminoglycoside-derived azide to generate substituted 1,2,3-triazoles, 1 (Scheme 1) (Vakulenko and Mobashery, 2003; Kolb and Sharpless, 2003). Click chemistry techniques involving aminoglycoside-based azides are explained in Quader et al., 2007, incorporated herein by reference.

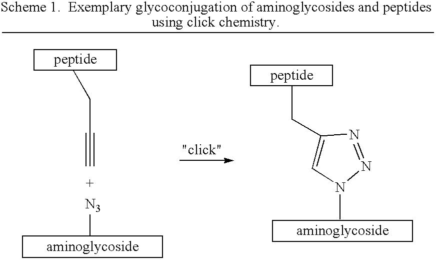

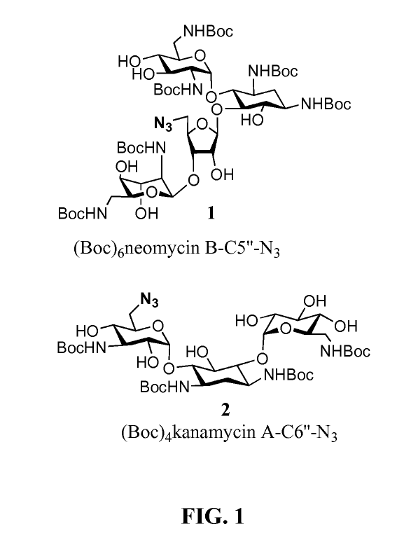

The single primary hydroxymethyl groups at the ribose moiety (5″-position) in neomycin B and at the 3-deoxy-3-amino-glucose moiety (6′-position) of kanamycin A were initially selected for introduction of the azido function. In order to be compatible with solution and solid phase peptide chemistry using the Fmoc-strategy, all remaining amino functions in neomycin B- and kanamycin A-derived azides need to be protected with an acid labile protecting groups in the form of CBz or Boc. The inventors selected Boc-protected compounds 1 and 2 as azide components due to the milder acidic cleavage conditions and literature precedents (Disney and Barrett, 2007; Quader et al., 2007). See FIG. 1.

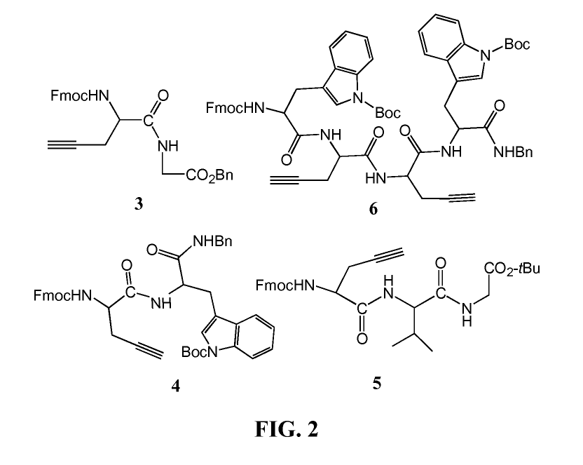

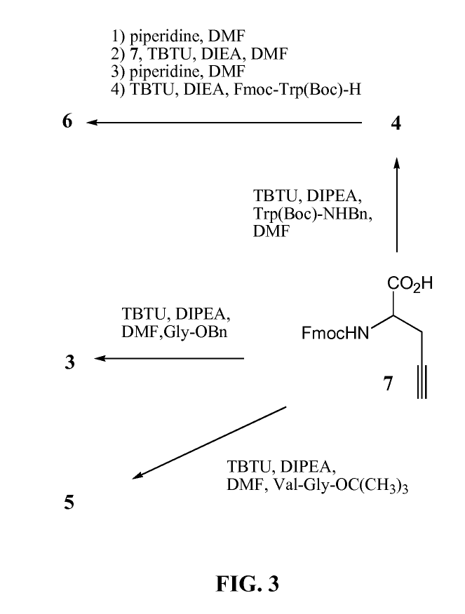

Syntheses of Peptides 3-6. (Boc)₆-neomycin-C5″-N₃ (1) and (Boc)₄-kanamycin-C6′-N₃ (2) were synthesized according to previously established procedures by Disney and Barrett (2007) and Quader et al. (2007). With both azides in hands the inventors then focused on the synthesis of a small collection of hydrophobic oligopeptides. The inventors selected dipeptides Fmoc-Pra-Gly-OBn (3) and Fmoc-Pra-Trp-NHBn (4), tripeptide Fmoc-Pra-Val-Gly-OBn (5) and tetrapeptide Fmoc-Trp(Boc)-Pra-Pra-Trp(Boc)-NHBn (6) a suitable candidates (FIG. 2). The peptides were prepared by solution phase peptide chemistry using 2-(1H-benzotriazole-1-yl)-1,1,3,3-tetra-methyluronium tetrafluoroborate (TBTU) as coupling reagent in DMF and the corresponding amino acid building blocks (FIG. 3). The terminal alkyne moiety was introduced into the peptides 3-6 via incorporation of the Fmoc-protected propargylglycine 7 (Schoen and Kisfaludy, 1986).

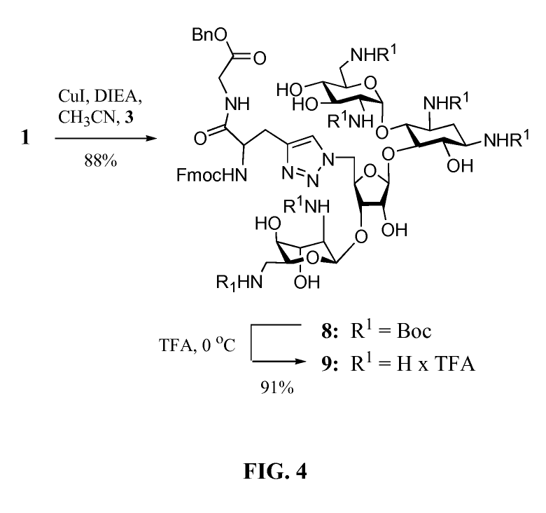

Solution Phase Syntheses of Neomycin-peptide Conjugates. Initially, the inventors studied the 1,3-dipolar cycloaddition reaction between (Boc)₆-neomycin B-C5″-N₃ (1) and peptide 3 using CuI and N,N-diisopropylethylamine in acetonitrile yielding neomycin-peptide conjugate (8) in 88% yield (FIG. 4). The formation of the triazole ring was confirmed by mass spectroscopy and NMR spectroscopy (see characterization data below). For instance, the ¹H NMR spectrum in C₅D₅N shows a vinylic proton appearing at δ=8.39 ppm as a singlet while the aromatic protons appeared at δ 7.30-7.80 ppm and the anomeric protons were noticed at δ 6.09, 5.77 and 5.40 ppm. In the ¹³C NMR spectrum in CD₃OD, the alkenic carbons of the triazole ring of compound 8 were observed at δ 126.5 (═C—H) and 145.0 (q) ppm while the anomeric carbons appeared at δ 111.2, 100.7 and 98.9 ppm. Deblocking of the aminoglycoside-peptide conjugate was achieved by exposure to 95% TFA at 0° C. for 3 min to produce the neomycin-peptidotriazole conjugate 9 as TFA salt in 91% yield (FIG. 4). In the ¹H-NMR spectrum of 9 (see characterization data below), the vinylic proton of the triazole ring appeared at δ 7.96 ppm in CD₃OD while the alkenic carbons were identified at δ 126.1 and 145.1 ppm in the ¹³C NMR in CD₃OD.

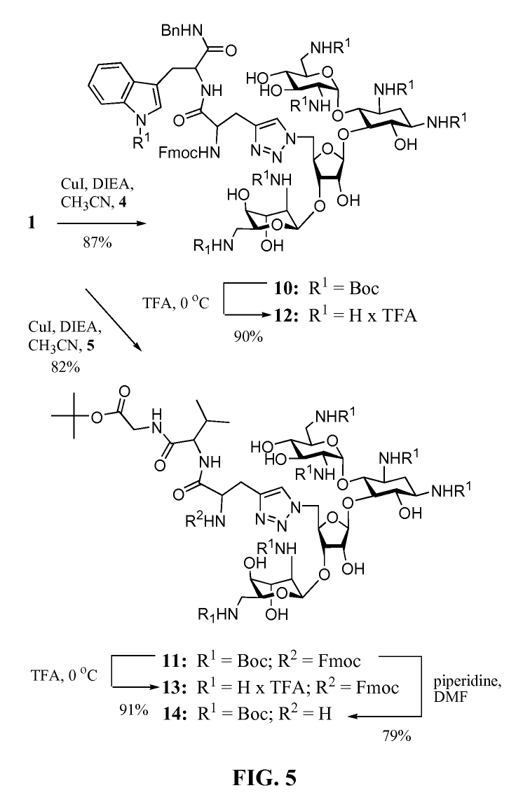

Glycoconjugation of (Boc)₆-neomycin-C5″-N₃ (1) to dipeptide 4 and tripeptide 5 was then explored (FIG. 5). CuI-mediated cycloaddition of azide 1 with peptides 4 and 5 produced the glycoconjugates 10 and 11 in 87% and 82% yield respectively. The structures of peptides 10 and 11 were confirmed by mass- and NMR spectroscopy (see characterization data below). In peptide 10 the alkenic proton appeared at δ 8.35 ppm while the aromatic protons were noticed in a region at δ 7.86-7.22 in C₅D₅N. In order to identify the alkenic proton of the triazole linkage in peptide 11 the inventors removed the Fmoc-group using piperidine in DMF to generate conjugate 14. ¹H and ¹³C NMR analysis of 14 confirmed the triazole linkage (see characterization data below). For instance, the ¹H NMR spectrum in CD₃OD showed the alkenic proton at δ 7.89 ppm as a singlet. Furthermore, two sp² hybridized C-atoms were identified at δ 126.6 and 144.9 (q) ppm in the ¹³C NMR.

Once the inventors confirmed the presence of the triazole tether or linkage in the peptide-conjugates 10 and 11, the inventors then investigated the regiochemistry of the cycloaddition. It is known that the thermal, non-catalyzed 1,3-dipolar cycloaddition of azides to alkynes is a regio-unspecific reaction generating a mixture of 1,4-and 1,5-substituted [1,2,3]-triazoles. However, copper(I)-catalyzed reaction between azides and alkynes yield selectively the 1,4-substituted triazole (Hoffman et al., 2002; Rodios, 1984; Tornøe et al., 2002; Cheshev et al., 2006). This could be confirmed through NMR spectroscopic analysis of compound 14. The triazole ring of compound 14 was confirmed from a HSQC experiment at 500 MHz in CD₃OD where the vinylic proton at δ=7.89 ppm showed carbon correlation to δ=126.6 ppm (see characterization data below). Subjection of the vinylic proton at δ 7.89 ppm to a one-dimensional ROESY experiment showed interproton effects (2.96% ROE) to one of the C-5″ methylene protons at δ 4.78 ppm. In addition, a three bond heteronuclear correlation between the methylene C-5″ at 126.6 ppm and the vinylic proton measured in a HMBC experiment confirmed the formation of a 1,4-substituted triazole ring (Hoffman et al., 2002; Rodios, 1984; Tornøe et al., 2002; Cheshev et al., 2006). This result is consistent with the empirical rule noted by Dondoni and Marra (Cheshev et al., 2006) that states that 1,4-substituted triazole rings display large Δ(δC4-δC5) values typically while 1,5-substituted triazoles exhibit negative or small values in various solvents. For instance, the inventors observed a Δ(δC4-δC5) value of 18.3 ppm in compound 14. Similar values were obtained for neomycin triazole conjugates 8 and 10. Deblocking of the Boc-protecting groups was achieved by exposure of conjugates 10 and 11 to TFA to produce compounds 12 and 13 respectively.

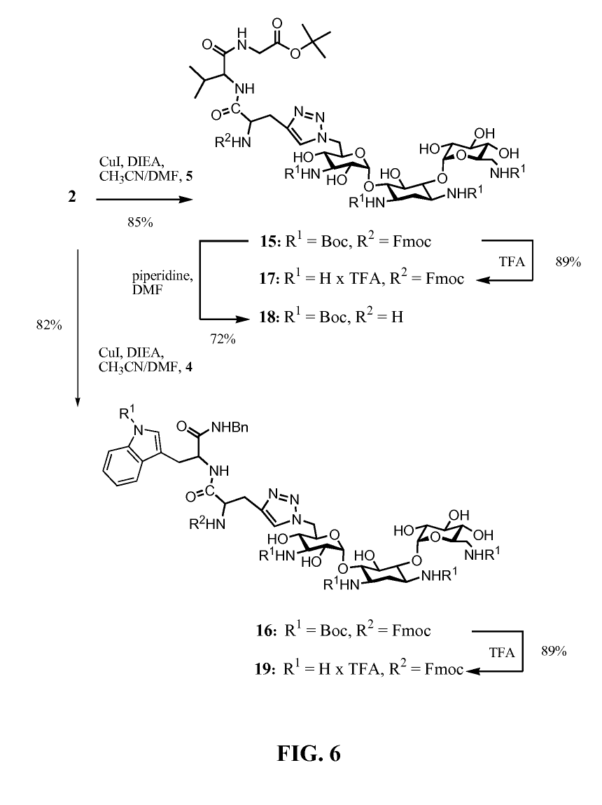

Next the inventors explored the cycloaddition of (Boc)₄-kanamycin A-C6″-N₃ (2) to peptides 4 and 5. Applying the same reaction protocol as previously applied to azide 1 the inventors obtained the kanamycin A-peptide conjugates 15 and 16 in 85% and 82% yield (FIG. 6). The formation of triazole tether in peptide 15 was confirmed by the appearance of a singlet at δ 7.83 ppm in the ¹H-NMR in CD₃OD that is attached to an sp²-hybridized carbon at δ 126.2 ppm in the ¹³C-NMR (see characterization data below). Similarly, the alkenic proton of conjugate 16 appeared at δ 8.04 ppm in the ¹H NMR and shows a correlation to a sp² hybridized C-atom in the HSQC experiment. The inventors also confirmed the presence of the triazole linkage in the Fmoc-deprotected conjugate 18. In this case the ¹H NMR spectroscopy showed the singlet appearing at δ 7.85 ppm which shows a correlation to a carbon atom at δ 125.9 ppm in the HSQC spectrum (see characterization data below).

To study the regioselectivity in the kanamycin A-based triazole formation, the inventors selected compound 18. Subjection of the vinylic proton at δ 7.83 ppm to a one-dimensional ROESY experiment showed interproton effects to the C-5″ methylene protons at δ 4.66 ppm (0.84% ROE) and 4.51 ppm (0.99% ROE). Both protons are correlated to a carbon atom at δ 52.6 ppm in an HSQC experiment and show a three bond heteronuclear correlation to the vinylic C—H carbon of the triazole unit as measured in an HMBC experiment. Moreover, the observed Δ(δC4-δC5) value of 18.5 corroborates the 1,4-substituted triazole ring in 18. Deprotection using TFA converted conjugates 15 and 16 into glycopeptides 17 and 19. The observed Δ(δC4-δC5)=18.9 for compound 16 indicates regioselective formation of the 1,4-disubstituted cycloaddition product.

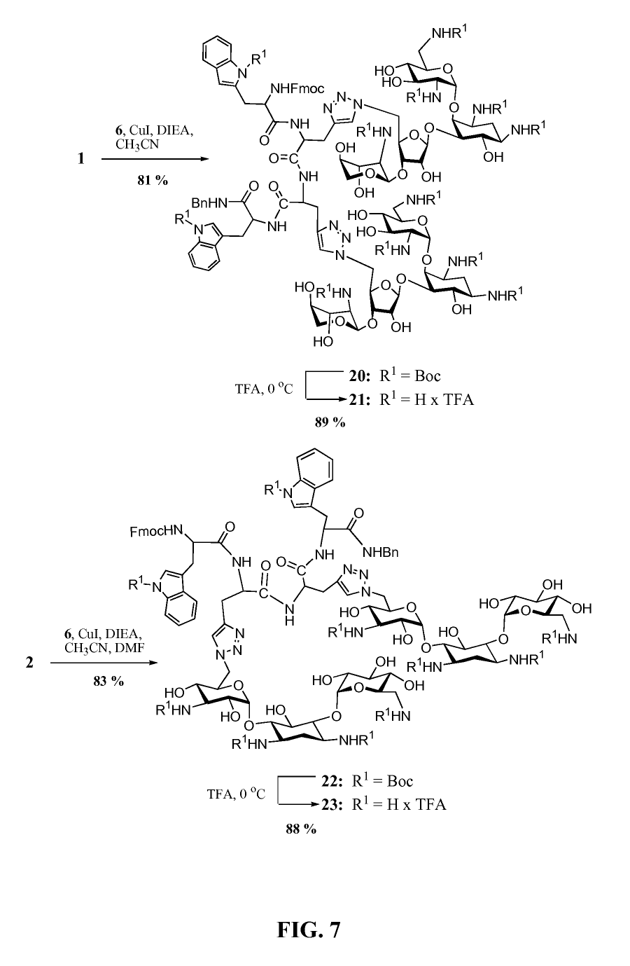

In order to explore the incorporation of multiple aminoglycoside moieties into a peptide component the inventors studied the cycloaddition of Fmoc-Trp-Pra-Pra-Trp-NHBn (6) to neomycin-based azide 1. Cu(I)-catalyzed cycloaddition using 2.5 equivalents of 1 and one equivalent of peptide 6 in a DMF/acetonitrile co-solvent produced bisaminoglycoside-peptide conjugate 20 in 81% isolated yield. FIG. 7. Thin-layer chromatography of the crude reaction mixture showed complete consumption of the peptide and formation of a single new spot. Reducing the amount of 1 resulted in incomplete conversion of peptide 6 as judged by thin layer chromatography. Applying the same reaction conditions to kanamycin A-based azide 2 produced bisaminoglycoside-peptide conjugate 22 in 83% isolated yield. FIG. 7. The observed chemical shifts for the alkenic protons and carbon atoms in compounds 20 and 22 are provided in Table 1. Treatment of compounds 20 and 22 with TFA produced deblocked bisaminoglycoside conjugates 21 and 23, respectively. FIG. 7. MS (ESI) analysis of 21 and 23 produced the expected molecular ions (M+H)⁺ at 2171.50 and 1910.68 for compounds 21 and 23 respectively (see characterization data below).

TABLE 1
Observed chemical shifts for alkenic protons
and carbon atoms in compounds 20 and 22.

| Compound | Triazole proton | Triazole carbon | Triazole carbon |
|---|---|---|---|
| 20 | 8.05 (s, 2 H) | 126.4 (x 2, CH) | 145.0 (x 2, C(q)) |
| 22 | 8.04 (s, 2 H) | 126.5 (x 2, CH) | 145.0 (x 2, C(q)) |

### Example 3

### Click-Based Glycoconjugation of Azide 1 with Solid Phase Supported Peptides

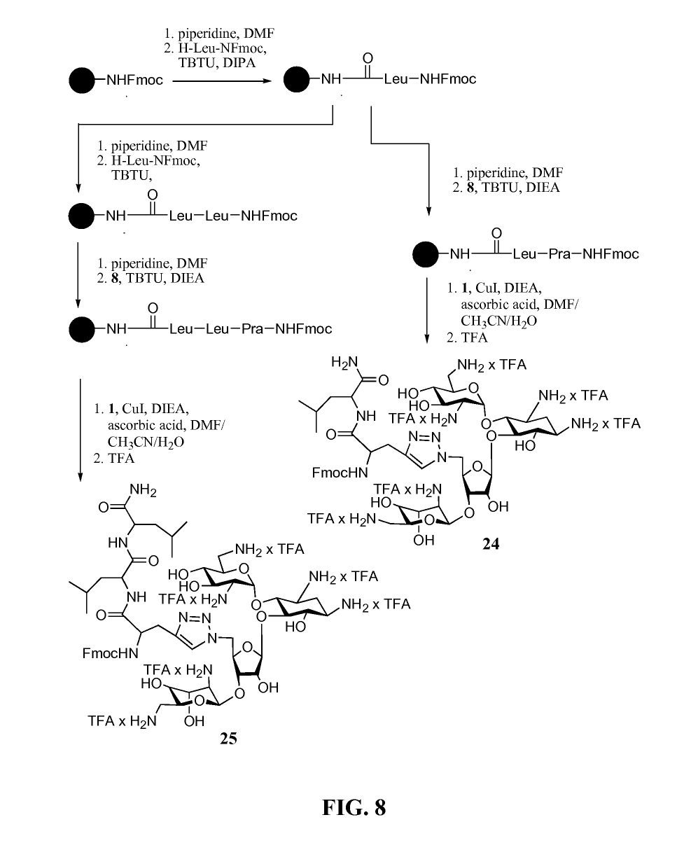

Solid phase peptide synthesis was performed on an Rink amide resin (Gogoi et al., 2007) using the Fmoc-strategy (Merrifield, 1986). Coupling of Fmoc-amino acids was performed with TBTU in DMF as solvent. The inventors selected dipeptide resin-Leu-Pra-NFmoc and tripeptide resin-Leu-Leu-Pra-NFmoc as model peptides and 1 as azide component to study the click-based cycloaddition reaction on solid support (FIG. 8). Exposure of azide 1 to resin-Leu-Pra-NFmoc and resin-Leu-Leu-Pra-NFmoc both in the presence of CuI, sodium ascorbate and DIEA in a ternary solvent containing DMF/water/CH₃CN afforded conjugates 24 and 25 after resin cleavage. The addition of sodium ascorbate improved the yield significantly suggesting that it is capable of stabilizing Cu in its +1 oxidation state (Rostovtsev et al., 2002; Sonogashira et al., 1975). Subsequently, the deblocked peptides were purified by HPLC and characterized by NMR (see characterization data below). In the ¹H NMR spectrum the triazole proton appeared at δ 7.99 ppm for both 24 and 25 and in the ¹³C NMR the alkenic carbon at δ 126.3 and 126.4 ppm and quarternay carbon at δ 145.1 and 145.2 ppm respectively. MS (ESI) analysis of 24 and 25 produced the expected molecular ions (M+H)⁺ at 1087.28 and 1200.56 respectively. The 1,4-di-substituted triazole ring in 24 and 25 was assigned based on large and positive Δ(δC4-δC5) values (Table 2).

TABLE 2
Observed Δ(δC4-δC5) values for various
aminoglycoside-peptide conjugates.

| Compound | Δ(δC4-δC5) in ppm^{a} |
|---|---|
| 8 | 18.5 |
| 10 | 18.9 |
| 11 | 18.7 |
| 14 | 18.3^{b} |
| 15 | 18.8 |
| 16 | 18.9 |
| 18 | 18.5^{b} |
| 20 | 18.9 |
| 22 | 18.7 |
| 24 | 18.8 |
| 25 | 18.8 |

^{a}assigned by HSQC;
^{b}assignment confirmed by HMBC and ROESY

### Example 4

### Characterization of 5″-Azido-1,3,2′,6′,2′″,6′″-hexa-N-(tert-butoxycarbonyl)-5″-deoxy-neomycin (1)

Yield=53%; IR (KBr disk) 2106.3 cm⁻¹ (N₃); ¹H NMR (300 MHz, CD₃OD): δ 5.47 (br s, 1H, anomeric), 5.16 (s, 1H, anomeric), 4.92 (s, 1H, anomeric), 4.53 (s, 1H), 4.33 (t, 1H, J=4.3 Hz), 4.28 (m, 1H), 4.03 (m, 1H), 3.92 (m, 2H), 3.76 (m, 3H), 3.56 (m, 5H), 3.38 (m, 3H), 3.32 (m, 3H), 3.21 (m, 2H), 1.98 (m, 1H), 1.67 (m, 1H), 1.46 (s, 54H); ¹³C NMR (75 MHz, CD₃OD): δ 159.0, 158.8, 158.4, 158.2, 158.1, 157.8, 111.6 (anomeric), 100.2 (anomeric), 98.7 (anomeric), 87.3, 80.9, 80.8, 80.5, 80.4, 79.8, 79.0, 78.5, 75.5, 75.2, 74.6, 73.4, 73.1, 72.9, 71.6, 69.1, 56.7, 53.7, 53.5, 52.5, 52.4, 51.4, 51.2, 42.7, 41.9, 35.5, 29.0, 28.9; EIMS: calcd for C₅₃H₉₃N₉NaO₂₄⁻ 1262.62 Found: 1262.62 (M+Na)⁺.

### Example 5

### Characterization of 6″-Azido-1,3,6′,3″-tetra-N-(tert-butoxycarbonyl)-6″-deoxy-kanamycin (2)

R_{f}: 0.36 (CH₂Cl₂/MeOH 12:1); IR (KBr disk) 2106.3 cm⁻¹ (N₃); ⁻¹H NMR (300 MHz, CD₃OD): δ 5.11 (br s, 1H), 5.10 (br s, 1H), 4.33 (d, 1H, J=9.8), 3.74 (d, 2H, J=9.8 Hz), 3.64 (t, 1H, J=9.2 Hz), 3.69 (dd, 1H, J=4.3, 11.5 Hz), 3.59 (m, 2H), 3.56 (d, 1H, J=2.5 Hz), 3.51 (d, 1H, J=2.5 Hz), 3.45 (dd, 2H, J=4.3, 11.5 Hz), 3.41 (d, 2H, J=3.8 Hz), 3.38 (t, 3H, J=3.6 Hz), 3.21 (t, 1H, J=9.4 Hz), 2.09 (br d, 1H, J=12.2 Hz), 1.55 (m, 1H), 1.47 (2s, 36H); ¹³C NMR (75 MHz, CD₃OD): δ 156.4, 155.6, 154.9, 101.4, 97.6, 84.6, 79.6, 78.0, 75.1, 72.7, 72.2, 71.2, 70.4, 70.1, 68.2, 55.5, 50.7, 49.9, 49.1, 40.8, 34.7, 27.9, 27.6; EIMS: calcd for C₃₈H₆₇N₄NaO₁₈³⁰ 932.44 Found: 932.38 [M+Na]^{+.}

### Example 6

### Peptide Syntheses and Characterization

General Procedures. Coupling of Fmoc-amino acid to amine groups of acid protected amino acid was performed with 2 equiv Fmoc-Amino Acid and 2 equiv TBTU and 4 equv. DIPEA in DMF at rt. Fmoc deprotection was achieved with 20% piperidine in DMF.

Fmoc-NH-Pra-Gly-O-Bn (3). ¹H NMR (300 MHz, CDCl₃): δ 7.76 (d, 2H, J=7.5 Hz), 7.59 (d, 2H, J=7.5 Hz), 7.40 (q, 4H, J=7.5 Hz), 7.33 (m, 5H), 5.58 (d, 1H, J=6.6 Hz), 4.45 (d, 2H, J=6.8 Hz), 4.40 (m, 2H), 4.23 (t, 1H, J=6.8 Hz), 4.10 (d, 2H, J=6.6 Hz), 2.82 (d, 1H, J=15.4 Hz), 2.72 (d, 1H, J=15.4 Hz), 2.07 (t, 1H, J=2.6 Hz); ¹³C NMR (75 MHz, CDCl₃): δ 169.8, 168.2, 155.9, 143.7, 141.3, 131.0, 128.7, 128.6, 127.8, 127.1, 125.0, 120.1, 79.0, 72.0, 67.4, 67.3, 53.3, 47.1, 41.5; EIMS: calcd. for C₂₉H₂₇N₂O₅⁺ 483.18 Found: 483.33 (M+H)⁺.

Fmoc-NH-Pra-Trp-NH-Bn (4). ¹H NMR (300 MHz, CD₃OD): δ 7.72 (d, 3H, J=7.5 Hz), 7.59 (d, 1H, J=7.9 Hz), 7.54 (d, 2H, J=7.4 Hz), 7.49 (br s, 1H), 7.35 (t, 3H, J=7.3 Hz), 7.26 (br t, 3H, J=7.5 Hz), 7.18 (br t, 2H, J=7.5Hz), 7.13 (m, 3H), 4.60 (t, 1H, J=6.9 Hz), 4.32 (m, 4H), 4.27 (t, 1H, J=6.6 Hz), 4.19 (t, 1H, J=6.9 Hz), 4.13 (d, 1H, J=6.7 Hz), 3.26 (q, 1H, J=7.2 Hz), 3.13 (q, 1H, J=7.5 Hz), 2.62 (d, 1H, J=3.9 Hz), 1.97 (m, 1H), 1.60 (s, 9H); ¹³C NMR (75 MHz, CD₃OD): 170.6, 170.3, 162.7, 156.1, 143.2, 143.1, 140.8, 137.0, 134.9, 129.9, 127.9, 127.2, 126.9, 126.7, 126.5, 124.5, 123.8, 122.2, 119.4, 118.5, 115.2, 114.6, 83.3, 78.4, 71.2, 66.7, 53.1, 46.5, 42.9, 36.0, 30.8, 27.5, 27.1; EIMS: calcd. for C₄₃H₄₃N₄O₆⁺ 711.31 Found: 711.33 (M+H)⁺.

Fmoc-NH-Pra-Val-Gly-O-^{t}Bu (5). ¹H NMR (300 MHz, CD₃OD): δ 7.74 (d, 2H, J=7.6 Hz), 7.57 (d, 2H, J=7.6 Hz), 7.37 (t, 2H, J=7.3 Hz), 7.27 (t, 2H, J=7.4 Hz), 4.52 (m, 1H), 4.40 (m, 2H), 4.20 (t, 1H, J=7.2 Hz), 3.93 (dd, 1H, J=4.4, 12.2 Hz), 2.96 (br s, 1H) 2.78 (br d, 1H, J=16.2 Hz), 2.63 (dd, 1H, J=5.5, 16.2 Hz), 2.18 (quintet, 1H, J=6.7 Hz), 2.11 (t, 1H, J=2.4 Hz), 1.46 (s, 9H), 1.38 d, 1H, J=2.8 Hz), 0.96 (t, 6H, J=7.1 Hz); ¹³C NMR (75 MHz, CD₃OD): 170.9, 170.2, 168.6, 156.1, 143.7, 141.3, 134.9, 129.2, 127.7, 127.0, 125.1, 120.0, 82.3, 58.6, 47.0, 41.9, 31.0, 27.9, 19.2, 18.1; EIMS: calcd. for C₃₁H₃₈N₃O₆⁺ 548.26 Found: 548.66 (M+H)⁺.

Fmoc-NH-Trp-Pra-Pra-Trp-NHBn (6). ¹H NMR (300 MHz, DMSO-d₆): δ 8.46 (br s, 1H), 8.44 (t, 1H, J=5.8 Hz), 8.23 (dd, 1H, J=7.9, 15.4 Hz), 8.05 (d, 1H, J=7.7 Hz), 8.02 (d, 1H, J=6.6 Hz), 7.66 (br s, 1H), 7.63 (d, 2H, J=7.5 Hz), 7.57 (d, 1H, J=7.7 Hz), 7.52 (d, 1H, J=8.7 Hz), 7.46 (d, 1H, J=7.7 Hz), 7.39 (d, 1H, J=7.2 Hz), 7.37 (t, 1H, J=6.7 Hz), 7.27 (t, 2H, J=9.1 Hz), 7.18 (dd, 2H, J=4.2, 7.5 Hz), 7.13 (m, 2H) 7.01 (m, 7H), 6.84 (br d, 1H, J=6.0 Hz), 4.66 (q, 1H, J=7.2 Hz), 4.45 (m, 3H), 4.28 (dd, 1H, J=5.8, 15.0 Hz), 4.20 (t, 1H, J=5.8 Hz), 4.18 (m, 2H), 4.09 (dd, 1H, J=4.8, 8.4 Hz), 3.12 (dd, 2H, J=5.3, 14.9 Hz), 3.02 (d, 1H, J=7.1 Hz), 2.96 (d, 1H, J=5.5 Hz), 2.92 (d, 1H, J=8.4 Hz), 2.79 (t, 1H, J=2.3 Hz), 2.74 (t, 1H, J=2.3 Hz), 2.56 (m, 3H), 1.40 (s, 9H), 1.34 (s, 9H); ¹³C NMR (75 MHz, DMSO-d₆): δ 171.9, 170.4, 170.2, 169.7, 162.6, 155.9, 149.3, 143.7, 140.7, 139.2, 134.7, 130.6, 128.2, 127.9, 127.0, 126.8, 125.6, 125.3, 124.1, 122.7, 120.4, 119.7, 119.5, 117.1, 116.3, 114.7, 83.7, 83.6, 80.5, 80.3, 73.3, 65.9, 54.5, 53.0, 51.8, 49.6, 42.2, 38.3, 35.9, 31.0, 28.3, 27.1; EIMS: calcd. for C₆₄H₆₆N₇O₁₀⁺ 1092.48 Found: 1092.55 (M+H)⁺.

General experimental procedure for the conjugation of aminoglycosides to peptides in solution phase. To a solution of alkyne (1 mmol) and azide (1 mmol) in acetonitrile (10 mL) were added CuI (0.2 mmol) and N,N-diisopropylethylamine (3 mmol) at room temperature, and the mixture was stirred for 1-2 h. At the end of the reaction as judged by TLC analysis, the reaction mixture was diluted using 25 mL of water and 10 mL of NH₄Cl, the aqueous layer was extracted with ethylacetate (3×50 mL), and the combined organic layer was washed with brine solution, dried over anhydrous sodium sulfate, and concentrated in vacuo to obtain a crude residue that was purified by flash chromatography (MeOH/CH₂Cl₂) to obtain the desired 1,4-disubstited 1,2,3-triazole as a white solid.

General procedure for removal of Boc group: Triazole derivative (0.050 mmol) was treated with 99% trifuoroacetic acid (4 mL) for 3 min at 0° C. The volatiles were removed in vacuo. The nonpolar residues were removed by washing with ether/methanol (1%) mixture and decanted the solvent to get triazole derivative as TFA salt.

Compound 8. Yield=88%; R_{f} 0.43 (MeOH/CH₂Cl₂ 1:14); ¹H NMR (300 MHz, Pyridine-d₅): δ 9.28 (br s, 1H), 9.20 (d, 1H J=7.3 Hz), 8.39 (br s, 1H, triazole-H), 7.83 (d, 2H, J=7.3 Hz), 7.68 (t, 2H, J=6.6 Hz), 7.40 (d, 2H, J=7.3 Hz), 7.38 (d, 2H, J=7.8 Hz), 7.36 (t, 1H, J=2.3 Hz), 7.37 (m, 5H), 6.09 (s, 1H, anomeric), 5.77 (br s, 1H, anomeric), 5.40 (br s, 1H, anomeric), 5.28 (m, 3H), 5.15 (d, 1H, J=3.6 Hz), 5.02 (d, 1H, J=14.4 Hz), 4.88 (d, 1H, J=14.4 Hz), 4.78 (br s, 1H), 4.70 (m, 2H), 4.61 (d, 1H, J=9.9 Hz), 4.52 (m, 2H), 4.48 (m, 2H), 4.44 (d, 2H, J=7.7 Hz), 4.37 (m, 2H), 4.33 (t, 1H, J=6.4 Hz), 4.18 (m, 2H), 4.07 (m, 4H), 3.96 (m, 1H), 3.86 (dd, 2H, J=5.7, 15.4 Hz), 3.80 (m, 1H), 3.68 (t, 1H, J=6.7 Hz), 3.61 (m, 1H), 3.30 (br s, 1H), 2.38 (d, 1H, J=10.6 Hz), 1.80 (d, 1H, J=10.6 Hz), 1.46 (s, 54H); ¹³C NMR (75 MHz, CD₃OD): δ 174.7, 171.2, 159.4, 158.9, 158.5, 158.3, 157.7, 145.3, 145.0, 142.6, 137.1, 131.5, 129.7, 128.9, 129.4, 126.5 (triazole), 121.1, 111.2 (anomeric), 100.7 (anomeric), 98.9 (anomeric), 85.3, 80.9, 80.8, 80.5, 80.3, 79.9, 75.8, 74.5, 73.1, 72.9, 71.6, 68.9, 68.3, 68.2, 52.9, 50.1, 51.2, 49.8, 49.5, 49.2, 48.9, 48.7, 48.4, 42.3, 36.1, 29.4, 29.0, 28.9; [α]_{D}²⁵=+32.0 (c 0.8, MeOH); EIMS: calcd. for C₈₂H₁₁₉N₁₁NaO₂₉⁺ 1744.81 Found: 1746.01 (M+Na)⁺. Anal. Calcd for C₈₂H₁₁₉N₁₁O₂₉ C, 50.16; H, 7.42; N, 10.77; Found: C, 50.49; H, 7.87; N, 10.89.

Compound 9. Yield=91%; R_{f} 0.21 (NH₄OH/MeOH/CH₂Cl₂, 2:5:5); ¹H NMR (300 MHz, CD₃OD): δ 7.96 (br s, 1H, triazole), 7.81 (d, 2H, J=7.5 Hz), 7.64 (d, 2H, J=6.9 Hz), 7.41 (t, 2H, J=7.5 Hz), 7.34 (m, 7H), 5.95 (d, 1H, anomeric, J=2.8 Hz), 5.41(d, 1H, anomeric, J=2.5 Hz), 5.38 (br s, 1H, anomeric), 4.72 (dd, 2H, J=5.5, 13.3 Hz), 4.55 (m, 3H), 4.45 (t, 1H, J=4.3 Hz), 4.37 (t, 1H, J=7.5 Hz), 4.31 (m, 1H), 4.22 (t, 2H, J=6.6 Hz), 4.16 (t, 2H, J=3.0 Hz), 4.09 (t, 2H, J=7.9 Hz), 4.02 (m, 3H), 3.85 (m, 2H), 3.70 (br s, 1H), 3.57 (t, 2H, J=9.6 Hz), 3.45 (m, 3H), 3.40 (m, 3H), 3.29 (m, 3H), 3.12 (m, 1H), 2.44 (br s, 1H), 2.06 (m, 1H); ¹³C NMR (75 MHz, CD₃OD): δ 173.9, 170.9, 158.3, 145.2, 145.1, 142.6, 136.9, 129.7, 129.4, 128.2, 126.1 (triazole), 121.1, 111.2, 96.8, 96.4, 86.8, 81.4, 76.7, 74.5, 73.9, 72.6, 72.2, 69.1, 68.0, 56.0, 52.9, 51.2, 42.3, 41.6, 29.9, 29.5. 28.9; [α]_{D}²⁵=+27.0 (c 0.03, MeOH); EIMS: calcd. for C₅₂H₇₁KN₁₁O₁₇⁺ 1160.47 Found: 1160.69 (M+K)⁺.

Compound 10. Yield=87%; R_{f} 0.39 (MeOH/CH₂Cl₂ 1:14); ¹H NMR (300 MHz, Pyridine-d₅): δ 9.73 (t, 1H, J=7.1 Hz), 9.68 (d, 1H, J=7.3 Hz), 9.11 (d, 1H, J=5.8 Hz), 8.35 (br s, 1H, triazole-H), 7.86 (br s, 1H), 7.83 (d, 2H, J=7.3 Hz), 7.80 (d, 1H, J=7.5 Hz), 7.72 (d, 2H, J=7.3 Hz), 7.66 (br s, 1H), 7.40 (m, 6H). 7.31 (d, 3H, J=7.3 Hz) 7.30 (d, 2H, J=7.3 Hz), 7.19 (m, 1H), 6.01 (br d, 1H, anomeric, J=3.3 Hz), 5.77 (br s, 1H, anomeric), 5.45 (br s, 1H, anomeric), 5.27 (t, 2H, J=6.9 Hz), 5.05 (m, 3H), 4.82 (m, 3H), 4.65 (m, 2H), 4.56 (m, 2H), 4.51 (m, 3H), 4.42 (t, 1H, J=9.4 Hz), 4.33 (t, 1H, J=7.3 Hz), 4.11 (m, 3H), 4.00 (m, 3H), 3.84 (m, 3H), 3.72 (d, 2H, J=7.2 Hz), 3.66 (dd, 2H, J=6.6, 14.3 Hz), 3.55 (dd, 1H, J=6.6, 14.3 Hz), 3.43 (m, 1H), 2.39 (d, 1H, J=11.7 Hz), 1.82 (m, 1H), 1.64 (br s, 9H), 1.55 (2s, 54H); ¹³C NMR (75 MHz, CD₃OD): δ 173.2, 173.1, 172.9, 159.2, 158.9, 158.5, 158.3, 158.1, 157.8, 151.0, 145.2, 145.1, 142.9, 139.2, 137.1, 131.5, 129.7, 129.4, 128.7, 128.3, 126.2 (triazole), 125.8, 123.9, 120.8, 120.4, 117.1, 115.9, 110.9 (anomeric), 100.4 (anomeric), 99.0 (anomeric), 84.7, 81.0, 80.5, 75.3, 74.3, 73.3, 72.6, 71.4, 68.9, 68.4, 56.5, 56.1, 55.0, 52.7, 44.0, 42.5, 42.0, 35.8, 28.9, 28.7, 28.4; [α]_{D}²⁵=+27.0 (c 0.95, MeOH); EIMS: calcd. for C₉₅H₁₃₃NaN₁₃O₃₀⁺ 1972.93 Found: 1973.71 (M+Na)⁺. Anal. Calcd for C₉₅H₁₃₃N₁₃O₃₀ C, 56.43; H, 7.33; N, 9.40; Found: C, 57.03; H, 7.56; N, 9.76.

Compound 12. Yield=90%; R_{f} 0.23 (NH₄OH/MeOH/CH₂Cl₂, 2:5:5); ¹H NMR (300 MHz, CD₃OD): δ 7.83 (d, 1H, J=7.5 Hz), 7.79 (br s, 1H), 7.76 (d, 1H, J=7.9 Hz), 7.63 (d, 1H, J=7.3 Hz), 7.57 (d, 2H, J=6.9 Hz), 7.42 (br d, 2H, J=7.5 Hz), 7.39 (t, 1H, J=3.8 Hz), 7.33 (br d, 1H, J=3.8 Hz), 7.29 (t, 1H, J=6.7 Hz), 7.19 (d, 1H, J=8.1 Hz) 7.12 (m, 5H), 6.99 (m, 2H), 5.97 (br s, 1H), 5.43 (d, 1H, J=3.5 Hz), 5.38 (br s, 1H), 4.69 (m, 4H), 4.56 (t, 1H, J=5.0 Hz), 4.46 (m, 3H), 4.33 (t, 2H, J=5.0 Hz), 4.30 (d, 1H, J=5.0 Hz), 4.23 (d, 1H, J=6.1 Hz), 4.18 (q, 2H, J=3.2 Hz), 4.12 (m, 2H), 4.04 (m, 4H), 3.96 (m, 1H), 3.82 (t, 1H, J=7.9 Hz), 3.72 (br s, 1H), 3.58 (t, 2H, J=9.3 Hz), 3.47 (m, 4H), 3.24 (m, 3H), 3.04 (m, 1H), 2.52 (m, 1H), 2.09 (m, 1H); ¹³C NMR (75 MHz, CD₃OD): δ 173.7, 173.3, 158.6, 145.1(q). 142.6, 139.2, 138.1, 130.0, 129.4, 129.2, 128.9, 128.7, 128.5, 128.2, 126.3 (triazole), 126.2, 122.7, 121.0, 119.5, 110.7 (anomeric), 97.0 (anomeric), 96.4 (anomeric), 81.6, 74.7, 73.9, 72.2, 69.4, 69.2, 69.1, 53.0, 51.2, 50.2, 44.3, 41.7, 30.5, 29.4, 28.7; [α]_{D}²⁵=+25.0 (c 0.45, MeOH); EIMS: calcd. for C₆₁H₇₉N₁₃NaO₁₆⁺ 1272.56 Found: 1272.57 (M+Na)⁺.

Compound 11. Yield=82%; R_{f} 0.37 (MeOH/CH₂Cl₂ 1:14); ¹H NMR (300 MHz, CD₃OD): δ 7.90 (br s, 1H), 7.80 (d, 2H, J=7.6 Hz), 7.62 (d, 2H, J=7.6 Hz), 7.39 (t, 2H, J=7.3 Hz), 7.36 (t, 2H, J=7.6 Hz), 5.40 (br s, 1H), 5.05 (br s, 1H), 4.96 (br s, 1H), 4.77 (m, 1H), 4.57 (m, 2H), 4.33 (m, 4H), 4.22 (t, 3H, J=7.2 Hz), 3.93 (m, 3H), 3.79 (m, 3H), 3.69 (dt, 1H, J=3.7, 9.9 Hz), 3.54 (t, 2H, J=9.5 Hz), 3.52 (m, 2H), 3.40 (m, 5H), 3.28 (m, 4H), 3.12 (m, 1H), 2.18 (quintet, 1H, J=6.2 Hz), 1.95 (t, 1H, J=14.2 Hz), 1.65 (m, 1H), 1.46 (4s, 63H), 1.00 (d, 3H, J=7.3 Hz), 0.98 (d, 3H, J=6.6 Hz); ¹³C NMR (75 MHz, CD₃OD): δ 173.8, 173.7, 170.4, 170.3, 159.3, 158.9, 158.4, 158.2, 157.9, 145.2, 145.1(q), 142.6, 128.9, 128.3, 126.4 (triazole), 121.1, 111.2 (anomeric), 100.6 (anomeric), 98.9 (anomeric), 85.4, 80.9, 80.7, 80.5, 80.3, 80.2, 79.6, 75.5, 74.5, 73.0, 72.9, 69.0, 68.4, 59.8, 56.6, 53.6, 52.7, 50.9, 50.1, 49.9, 43.0, 42.0, 35.9, 31.9, 29.0, 28.9, 28.8, 28.5, 20.0, 18.4; [α]_{D}²⁵=+20.0 (c 1.35, MeOH); EIMS: calcd. for C₈₄H₁₃₀N₁₂NaO₃₀ 1809.89 Found: 1809.79 (M+Na)⁺. Anal. Calcd for C₈₄H₁₃₀N₁₂O₃₀ C, 52.93; H, 7.72; N, 10.73; Found: C, 53.21; H, 7.93; N, 11.01.

Compound 13. Yield=91%; R_{f} 0.20 (NH₄OH/MeOH/CH₂Cl₂, 2:5:5); ¹H NMR (300 MHz, CD₃OD): δ 7.90 (br s, 1H), 7.76 (d, 2H, J=7.8 Hz), 7.62 (d, 2H, J=6.5 Hz), 7.37 (t, 2H, J=7.3 Hz), 7.28 (t, 2H, J=6.5 Hz), 5.98 (br s, 1H), 5.40 (br s, 1H), 5.33 (br s, 1H), 4.82 (m, 1H), 4.72 (m, 1H), 4.45 (m, 1H), 4.38 (t, 2H, J=7.2 Hz), 4.32 (m, 3H), 4.17 (m, 2H), 4.13 (d, 1H, J=10.2 Hz), 4.04 (m, 2H), 3.93 (m, 2H), 3.85 (d, 1H, J=9.1 Hz), 3.71 (m, 2H), 3.59 (d, 1H, J=9.1 Hz), 3.45 (m, 6H), 3.40 (m, 1H), 3.32 (m, 3H), 3.25 (m, 2H), 2.48 (d, 1H, J=10.4 Hz), 2.08 (m, 2H), 0.98 (s, 6H); ¹³C NMR (75 MHz, CD₃OD): δ 174.0, 173.8, 173.1, 158.3, 145.2, 142.6, 128.8, 128.2, 126.2 (triazole), 120.9, 111.4 (anomeric), 96.8 (anomeric), 96.2 (anomeric), 86.5, 81.3, 77.9, 76.6, 74.7, 74.0, 73.8, 73.0, 72.6, 72.2, 72.0, 69.5, 69.4, 69.1, 68.2, 60.2, 55.8, 55.5, 54.9, 52.9, 51.3, 50.3, 41.7, 35.8, 32.0, 29.4, 19.7, 18.6; [α]_{D}²⁵=+28.0 (c 1.2, MeOH); EIMS: calcd. for C₅₄H₈₄N₁₂O₁₈⁺ 1188.59 Found: 1188.42 (M+H)⁻.

Compound 14. Yield=79%; R_{f} 0.18 (MeOH/CH₂Cl₂ 3:14); ¹H NMR (300 MHz, CD₃OD): δ 7.89 (br s, 1H), 5.48 (br s, 1H), 5.10 (br s, 1H), 4.96 (br s, 1H), 4.78 (m, 1H), 4.30 (d, 1H, J=5.9 Hz), 4.18 (m, 3H), 4.00 (d, 1H, J=5.5 Hz), 3.95 (d, 1H, J=6.6 Hz), 3.93 (t, 1H, J=3.7 Hz), 3.85 (m, 1H), 3.79 (m, 4H), 3.69 (dt, 1H, J=3.7, 9.9 Hz), 3.54 (t, 2H, J=9.0 Hz), 3.52 (m, 2H), 3.40 (d, 2H, J=8.5 Hz), 3.36 (m, 4H), 3.27 (d, 1H, J=9.5 Hz), 3.22 (m, 2H), 3.03 (dd, 1H, J=7.8, 14.4 Hz), 2.18 (quintet, 1H, J=6.2 Hz), 1.95 (m, 1H), 1.65 (m, 1H), 1.46 (4s, 64H), 1.00 (d, 3H, J=7.3 Hz), 0.98 (d, 3H, J=7.4 Hz); ¹³C NMR (75 MHz, CD₃OD): δ 176.4, 173.9, 170.4, 170.3, 159.1, 158.9, 158.4, 158.2, 158.1, 157.9, 144.9 (triazole), 126.6 (triazole), 111.6 (anomeric), 100.6 (anomeric), 98.6 (anomeric), 86.6, 82.9, 80.9, 80.7, 80.5, 80.3, 79.0, 75.4, 74.8, 74.5, 72.9, 72.6, 71.6, 69.0, 59.6, 56.6, 56.0, 53.6, 52.7, 50.9, 43.0, 42.3, 42.1, 35.9, 31.9, 29.0, 28.9, 28.8, 28.5, 19.9, 18.4; [α]_{D}²⁵=+25.0 (c 1.0, MeOH); EIMS: calcd. for C₆₉H₁₂₂N₁₂NaO₂₈ ⁺ 1587.82 Found: 1588.50 (M+Na)⁺.

Compound 15. Yield=85%; R_{f} 0.40 (MeOH/CH₂Cl₂ 1:12); ¹H NMR (300 MHz, CD₃OD): δ 7.83 (br s, 1H), 7.80 (d, 2H, J=7.1 Hz), 7.65 (m, 2H), 7.40 (t, 2H, J=7.7 Hz), 7.32 (t, 2H, J=7.7 Hz), 5.08 (br s, 2H), 4.64 (d, 1H, J=7.0 Hz), 4.56 (q, 1H, J=6.9 Hz), 4.36 (m, 2H), 4.30 (d, 1H, J=6.4 Hz), 4.23 (t, 1H, J=6.4 Hz), 3.91 (q, 2H, J=17.1 Hz), 3.72 (m, 3H), 3.66 (t, 2H, J=9.4 Hz), 3.51 (m, 5H), 3.40 (m, 3H), 3.23 (m, 3H), 3.10 (m, 1H), 3.01 (t, 1H, J=9.6 Hz), 2.17 (q, 1H, J=6.2 Hz), 1.97 (m, 1H), 1.68 (m, 1H), 1.46 (3s, 45H), 1.00 (d, 3H, J=7.3 Hz), 0.98 (d, 3H, J=7.3 Hz); ¹³C NMR (75 MHz, CD₃OD): δ 173.7, 173.4, 170.2, 159.4, 159.3, 158.3, 157.8, 157.7, 145.2, 145.1 (triazole), 142.6 142.5, 128.9, 128.3, 126.3 (triazole), 121.0, 102.8 (anomeric), 99.4 (anomeric), 85.6, 82.9, 80.6, 80.4, 80.2, 76.8, 74.5, 73.9, 72.4, 71.9, 71.8, 71.7, 68.4, 60.0, 57.2, 56.2, 50.8, 42.9, 41.9, 36.2, 31.9, 30.7, 29.2, 28.9, 28.8, 28.4, 19.9, 18.5; [α]_{D}²⁵=+20.0 (c 1.4, MeOH); EIMS: calcd. for C₆₉H₁₀₄N₁₀NaO₂₄⁺ 1479.71 Found: 1479.51 (M+Na)⁺. Anal. Calcd for C₆₉H₁₀₄N₁₀O₂₄ C, 56.86; H, 7.19; N, 9.61; Found: C, 57.16; H, 7.53; N, 9.44.

Compound 17. Yield=89%; R_{f} 0.21 (NH₄OH/MeOH/CH₂Cl₂, 2:5:5); ¹H NMR (300 MHz, CD₃OD): δ 8.00 (br s, 1H), 7.77 (d, 2H, J=7.1 Hz), 7.62 (d, 2H, J=6.5 Hz), 7.37 (t, 2H, J=7.3 Hz), 7.29 (t, 2H, J=7.3 Hz), 5.43 (d, 1H, J=2.9 Hz), 5.09 (d, 1H, J=2.9 Hz), 4.35 (m, 1H), 4.21 (m, 1H), 4.07 (ABq, 2H, J=9.6 Hz), 3.85 (dd, 3H, J=9.2, 17.1 Hz), 3.76 (d, 1H, J=6.6 Hz), 3.69 (d, 1H, J=9.2 Hz), 3.64 (m, 2H), 3.58 (m, 4H), 3.46 (t, 1H, J=5.6 Hz), 3.43 (m, 3H), 3.41 (m, 3H), 3.30 (m, 1H), 3.23 (t, 2H, J=9.6 Hz), 3.01 (m, 1H), 2.52 (m, 1H), 2.14 (m, 2H), 0.98 (s, 6H); ¹³C NMR (75 MHz, CD₃OD): δ 173.9, 170.2, 158.5, 145.1, 142.6, 128.9, 128.3, 126.3 (triazole), 121.0, 102.4 (anomeric), 96.4 (anomeric), 85.7, 80.9, 74.6, 73.5, 73.3, 72.9, 70.9, 70.5, 70.2, 68.1, 56.7, 56.6, 52.4, 51.9, 42.1, 31.9, 28.9, 19.7, 18.5; [α]_{D}²⁵=+45.0 (c 0.6, MeOH); EIMS: calcd. for C₄₉H₇₂N₁₀O₁₆ 1057.52 Found: 1057.51 (M+Na)⁺.

Compound 18. Yield=72%; R_{f} 0.16 (MeOH/CH₂Cl₂ 1:9); ¹H NMR (300 MHz, CD₃OD): δ 7.85 (br s, 1H), 5.11 (d, 1H, J=2.8 Hz), 5.06 (d, 1H, J=3.1 Hz), 4.66 (d, 1H, J=13.4 Hz), 4.50 (t, 1H, J=8.2 Hz), 4.49 (m, 1H), 4.24 (d, 1H, J=6.4 Hz), 3.91 (q, 2H, J=17.1 Hz), 3.79 (m, 3H), 3.65 (t, 1H, J=9.4 Hz), 3.54-3.40 (m, 8H), 3.19 (m, 2H), 3.11 (t, 1H, J=5.3 Hz), 3.06 (m, 2H), 2.11 (quintet, 1H, J=6.6 Hz), 2.02 (dt, 1H, J=3.3, 12.1 Hz), 1.68 (m, 1H), 1.46 (4s, 45H), 1.00 (d, 3H, J=5.9 Hz), 0.98 (d, 3H, J=6.5 Hz); ¹³C NMR (75 MHz, CD₃OD): δ 173.9, 170.2, 159.4, 159.3, 158.3, 157.7, 144.4 (triazole), 125.9 (triazole), 102.9, 99.7, 85.8, 82.9, 80.6, 80.4, 80.2, 76.9, 74.4, 73.9, 72.4, 60.0, 57.2, 55.7, 52.6, 52.3, 50.8, 42.9, 41.9, 32.0, 31.9, 29.0, 28.9, 28.8, 28.7, 28.4, 28.3, 19.8, 18.5; [α]_{D}²⁵=+20.0 (c 0.4, MeOH); EIMS: calcd. for C₅₄H₉₄N₁₀NaO₂₂⁻ 1257.65 Found: 1257.51 (M+Na)⁺.

Compound 16. Yield=82%; R_{f} 0.36 (MeOH/CH₂Cl₂ 1:12); ¹H NMR (300 MHz, Pyridine-d₅): δ 9.83 (d, 1H, J=7.8 Hz), 9.68 (t, 1H, J=6.2 Hz), 9.14 (d, 1H, J=8.1 Hz), 8.33 (d, 1H, J=8.1 Hz), 8.04 (s, 1H, triazole), 7.86 (d, 2H, J=7.5 Hz), 7.72 (t, 4H, J=6.9 Hz), 7.44 (t, 2H, J=7.2 Hz), 7.34 (m, 3H), 7.26 (m, 4H), 7.18 (m, 3H), 5.68 (s, 1H), 5.64 (s, 1H), 5.49 (q, 1H, J=6.9 Hz), 5.24 (ABq, 2H, J=6.9 Hz), 5.00 (m, 1H), 4.91 (m, 1H), 4.84 (m, 1H), 4.78 (d, 1H, J=5.5 Hz), 4.66 (d, 1H, J=4.3 Hz), 4.61 (m, 1H), 4.56 (m, 2H), 4.46 (m, 1H), 4.31 (t, 2H, J=6.9 Hz), 4.06 (m, 4H), 3.93 (t, 2H, J=8.1 Hz), 3.80 (br t, 1H, J=8.4 Hz), 3.70 (q, 2H, J=7.2 Hz), 3.66 (d, 1H, J=7.2 Hz), 3.62 (t, 1H, J=3.2 Hz), 3.59 (t, 1H, J=6.4 Hz), 3.46 (d, 1H, J=6.0 Hz), 3.43 (d, 1H, J=6.4 Hz), 2.70 (d, 1H, J=11.7 Hz), 1.86 (m, 1H), 1.55 (4s, 45H); ¹³C NMR (75 MHz, CD₃OD): δ 173.2, 172.9, 159.2, 158.2, 157.4, 150.9, 145.2, 145.1, 142.9, 139.4, 136.7, 131.7, 129.4, 128.8, 128.4, 126.2 (triazole), 125.7, 121.3, 120.4, 117.1, 116.1, 102.9 (anomeric), 99.3 (anomeric), 84.8, 80.4, 76.5, 75.3, 73.9, 72.6, 71.5, 68.0, 57.3, 56.5, 54.9, 52.4, 50.8, 44.5, 41.9, 36.1, 29.3, 28.8, 28.5; [α]_{D}²⁵=+23.0 (c 0.75, MeOH); EIMS: C₈₁H₁₁₀N₁₁O₂₄⁺ 1621.75 Found: 1622.04 (M+H)⁺. Anal. Calcd for C₆₉H₁₀₄N₁₀O₂₄ C, 60.02; H, 6.78; N, 9.51; Found: C, 60.56; H, 6.98; N, 9.33.

Compound 19. Yield=89%; R_{f} 0.18 (NH₄OH/MeOH/CH₂Cl₂ 2:5:5); ¹H NMR (300 MHz, CD₃OD): δ 7.99 (s, 1H), 7.81(d, 2H, J=7.3 Hz), 7.80 (m, 4H), 7.78 (t, 2H, J=6.9 Hz), 7.53 (d, 3H, J=8.1 Hz), 7.47 (m, 7H), 6.03 (br s, 1H), 5.73 (br s, 1H), 4.68 (m, 2H), 4.43 (m, 2H), 4.27 (m, 5H), 4.07 (m, 3H), 3.81 (m, 4H), 3.65 (m, 3H), 3.43 (m, 6H), 3.28 (m, 3H, J=9.7 Hz), 2.51 (m, 1H), 1.93 (m, 1H); ¹³C NMR (75 MHz, CD₃OD): δ 173.8, 162.0, 158.5, 145.2, 142.6, 139.2, 138.0, 129.5, 128.9, 128.3, 126.3 (triazole), 124.9, 122.7, 121.0, 120.2, 119.4, 117.9, 114.0, 112.6, 110.6, 102.1 (anomeric), 97.1 (anomeric), 85.7, 80.6, 74.2, 73.9, 73.3, 72.8, 70.5, 69.9, 68.7, 68.4, 56.7, 55.9, 52.3, 51.6, 44.3, 42.4, 29.0, 28.9; [α]_{D}²⁵=+34.0 (c 0.35, MeOH); EIMS: calcd. for C₅₆H₇₀N₁₁O₁₄⁺ 1120.51 Found: 1120.57 (M+H)⁺.

Compound 20. Yield=81%; R_{f} 0.31 (MeOH/CH₂Cl₂ 1:10); ¹H NMR (300 MHz, Pyridine-d₅): 9.81 (d, 1H, J=7.7 Hz), 9.68 (t, 1H, J=5.7 Hz), 9.11 (d, 1H, J=7.9 Hz), 8.31 (d, 1H, J=7.7 Hz), 8.05 (s, 2H), 7.85 (d, 2H, J=7.3 Hz), 7.80 (d, 1H, J=4.5 Hz), 7.74 (t, 5H, J=7.7 Hz), 7.38 (t, 3H, J=7.3 Hz), 7.29 (m, 4H) 7.24 (m, 5H), 7.14 (m, 3H), 6.10 (br s, 2H), 5.95 (br s, 2H), 5.63 (br s, 1H), 5.58 (br s, 2H), 5.44 (ABq, 2H, J=6.0 Hz), 5.21 (ABq, 3H, J=6.7 Hz), 4.92 (m, 4H), 4.80 (dd, 2H, J=5.8, 14.3 Hz), 4.66 (dd, 2H, J=5.3, 15.4 Hz), 4.56 (m, 10H), 4.30 (t, 3H, J=6.9 Hz), 4.24 (br s, 2H), 3.99 (m, 10H), 3.88 (m, 5H), 3.79 (m, 5H), 3.66 (m, 8H), 3.45 (dd, 3H, J=8.3, 14.3 Hz), 3.13 (ABq, 1H, J=6.4 Hz), 2.68 (br d, 2H, J=12.2 Hz), 1.82 (m, 2H), 1.47 (8s, 126H); ¹³C NMR (75 MHz, CD₃OD): δ 173.0, 172.9, 172.7, 159.2, 158.9, 158.4, 158.2, 157.9, 150.7, 145.2, 145.0. 144.6, 142.4, 139.3, 136.7, 131.7, 129.4, 128.4, 126.3 (triazole), 126.4 (triazole), 125.8, 123.9, 120.3, 117.1, 116.1, 111.4 (anomeric), 111.3 (anomeric), 100.7 (anomeric), 100.1 (anomeric), 99.7 (anomeric), 98.7 (anomeric), 84.7, 80.9, 80.6, 75.5, 74.5, 73.1, 72.7, 72.4, 71.6, 70.5, 68.9, 68.5, 56.6, 55.9, 53.7, 51.1, 44.4, 43.9, 42.8, 42.3, 41.8, 35.9, 28.9, 28.6, 28.4; [α]_{D}²⁵=+22.0 (c 0.5, MeOH); MALDITOF: calcd. for C₁₇₀H₂₅₁N₂₅O₅₈: 3570.74; 1808.35 Found: 1809.71 (M/2+Na)⁺. Anal. Calcd for C₁₇₀H₂₅₁N₂₅O₅₈ C, 57.15; H, 7.08; N, 9.80; Found: C, 57.55; H, 7.39; N, 9.65.

Compound 21. Yield=89%; R_{f} 0.25 (NH₄OH/MeOH/CH₂Cl₂, 2:7:5); ¹H NMR (500 MHz, CD₃OD): δ 7.89 (br s, 1H), 7.76 (d, 2H, J=6.9 Hz), 7.72 (br s, 1H), 7.55 (m, 2H), 7.46 (m, 2H), 7.34 (m, 4H), 7.17 (m, 6H), 7.09 (m, 3H), 7.04 (m, 4H), 6.00 (br s, 1H), 5.95 (br s, 1H), 5.52 (m, 1H), 5.42 (br s, 1H), 5.38 (br s, 1H), 5.36 (br s, 1H), 5.31 (br s, 1H), 4.64 (m, 5H), 4.47 (m, 4H), 4.28 (m, 5H), 4.21 (m, 2H), 4.13 (m, 4H), 4.07 m, 3H), 4.01 (m, 3H), 3.94 (m, 2H), 3.83 (m, 4H), 3.67 (m, 2H), 3.55 (m, 3H), 3.49 (m, 4H), 3.42 (m, 6H), 3.36 (m, 4H), 3.22 (m, 7H), 3.06 (m, 2H), 2.43 (m, 2H), 2.04 (m, 2H); ¹³C NMR (75 MHz, CD₃OD): δ 175.3, 173.9, 173.2, 172.6, 158.8, 145.6, 145.7, 142.5, 142.4, 139.1, 138.4, 132.0, 131.4, 129.4, 128.9, 128.3, 126.3 (triazole), 126.1 (triazole), 125.1, 124.9, 122.7, 120.9, 120.1, 119.3, 119.1, 112.6 (anomeric), 110.7 (anomeric), 97.2 (anomeric), 96.9 (anomeric), 96.3 (anomeric), 96.1 (anomeric), 86.5, 81.3, 77.9, 77.4, 76.5, 75.6, 74.6, 74.1, 72.9, 72.1, 70.7, 69.2, 68.1, 58.3, 57.8, 55.8, 55.4, 52.9, 49.9, 44.6, 43.6, 41.9, 41.7, 29.1, 28.6; [α]_{D}²⁵=+23.0 (c 0.5, MeOH); EIMS: calcd. for C₁₀₀H₁₄₀N₂₅O₃₀⁺ 2171.02 Found: 2171.50 (M+H)⁺.

Compound 22. Yield=83%; R_{f} 0.15 (MeOH/CH₂Cl₂ 1:9); ¹H NMR (500 MHz, Pyridine-d₅): δ 8.04 (br s, 2H), 7.82 (d, 2H, J=6.1 Hz), 7.79 (br d, 3H, J=7.3 Hz), 7.64 (d, 1H, J=7.3 Hz), 7.40 (t, 3H, J=7.3 Hz), 7.38 (m, 5H), 7.26 (m, 7H), 7.00 (m, 2H), 5.62 (br s, 3H), 5.47 (m, 1H), 5.22 (s, 1H), 5.17 (m, 3H), 5.01 (m, 4H), 4.80 (m, 2H), 4.49 (q, 4H, J=8.7 Hz), 4.32 (m, 2H), 4.21 (m, 3H), 4.09 (m, 4H), 3.99 (m, 8H), 3.87 (m, 9H), 3.70 (m, 6H), 3.59 (m, 4H), 3.39 (m, 1H), 2.72 (m, 1H), 2.20 (m, 1H), 1.93 (m, 2H), 1.55 (4s, 90H); ¹³C NMR (75 MHz, CD₃OD): δ 173.1, 159.5, 159.1, 158.5, 158.0, 157.7, 151.2, 146.4, 145.2, 145.0, 142.5, 139.3, 136.8, 131.9, 131.2, 130.9, 129.6, 129.2, 128.7, 128.4, 126.5 (triazole), 126.4 (triazole), 125.7, 125.4, 124.0, 121.1, 120.5, 117.4, 116.1, 103.0 (anomeric), 102.8 (anomeric), 99.9 (anomeric), 99.8 (anomeric), 85.8, 84.7, 80.9, 80.4, 80.2, 77.0, 74.7, 74.1, 72.6, 72.0, 70.8, 68.6, 66.5, 63.7, 61.5, 57.1, 54.9, 54.1, 52.2, 50.9, 44.3, 42.1, 36.1, 31.0, 28.9, 28.5; [α]_{D}²⁵=+33.0 (c 0.6, MeOH); MALDITOF: calcd. for (C140H199N₂₁O₄₆/₂+Na)⁺ 1478.38 Found: 1478.64 (M/2+Na)⁺. Anal. Calcd for C₁₄₀H₁₉₉N₂₁O₄₆ C, 57.74; H, 6.89; N, 10.10; Found: C, 58.02; H, 7.08; N, 10.52.

Compound 23. Yield=88%; R_{f} 0.22 (NH₄OH/MeOH/CH₂Cl₂ 2:7:5); ¹H NMR (500 MHz, CD₃OD): δ 8.04 (br s, 2H), 7.75 (d, 2H, J=7.4 Hz), 7.70 (m, 1H), 7.60 (m, 2H), 7.49 (t, 2H, J=7.8 Hz), 7.41 (d, 1H, J=8.7 Hz), 7.35 (d, 2H, J=8.5 Hz), 7.31 (m, 3H), 7.19 (m, 4H), 7.11 (m, 3H), 7.00 (m, 3H), 5.45 (s, 1H), 5.43 (s, 1H), 5.05 (s, 1H), 5.10 (m, 1H), 5.01 (s, 1H), 4.72 (d, 2H, J=8.7 Hz), 4.64 (q, 3H, J=8.7 Hz), 4.50 (m, 2H), 4.36 (m, 4H), 4.31 (m, 1H), 4.25 (m, 2H), 4.21 (m, 2H), 4.12 (t, 1H, J=7.3 Hz), 4.03 (m, 4H), 3.90 (m, 1H), 3.83 (m, 3H), 3.74 (m, 5H), 3.63 (m, 6H), 3.43 (m, 5H), 3.25 (t, 2H, J=9.5 Hz), 3.16 (m, 4H), 3.10 (m, 1H), 3.02 (t, 2H, J=9.5 Hz), 2.50 (m, 2H), 2.30 (m, 1H), 1.96 (m, 1H); ¹³C NMR (75 MHz, CD₃OD): δ 175.2, 173.7, 172.9, 158.6, 145.2, 145.1, 142.7, 139.2, 137.9, 129.5, 129.1, 128.3, 126.2 (triazole), 124.8, 122.5, 120.8, 120.0, 119.4, 112.5, 110.7, 102.2 (anomeric), 102.0 (anomeric), 98.1 (anomeric), 97.0 (anomeric), 85.3, 80.9, 74.1, 73.1, 70.6, 69.8, 69.1, 68.6, 68.2, 56.5, 54.9, 52.7, 51.9, 51.4, 44.0, 42.5, 29.0, 28.8; [α]_{D}²⁵=+36.0 (c 0.035, MeOH); EIMS: C₉₀H₁₂₀N₂₁O₂₆⁺ 1910.87 Found: 1910.68 (M+H)⁻.

General procedure for solid phase peptide synthesis. MBHA resin (0.66 mmol/g, 1 eq) was pre-swollen in DMF. 20% piperidine in DMF was added to them and the suspension was stirred at room temperature for 1 h. The resin was washed with DCM, DMF and dried. Coupling of Fmoc-amino acid to amino groups was performed with 3 equiv Fmoc-AAcid and 3 equiv TBTU, 3 equv. DIPEA in DMF. Fmoc deprotection was effected with 20% piperidine in DMF for 2×30 min followed by washing of the resin six times with DMF. The resin was washed six times with the appropriate solvent between each reaction step. A small portion of the resin was subjected with TFA and detected by mass spectra. Then deprotection and coupling was performed in similar way.

General Procedures for solid phase click reaction. The click reaction was furnished with copper (I) iodide (17 eq), sodium ascorbate (20 eq), DIPEA (30 eq.), DMF-water-acetonitrile at rt for 5 days.

General procedure for resin cleavage. The peptide-triazole was cleaved from resin with 95% TFA. The resin was washed with water and methanol. All the solvents were collected and concentrated. The crude reaction mixture was washed with methanol/ether (1%).

Compound 24. Yield=52%; R_{f} 0.16 (NH₄OH/MeOH/CH₂Cl₂, 2:5:7); ¹H NMR (300 MHz, CD₃OD): δ 7.99 (br s, 1H), 7.80 (d, 2H, J=7.3 Hz), 7.65 (d, 2H, J=7.6 Hz), 7.39 (t, 2H, J=7.3 Hz), 7.31 (t, 2H, J=7.3 Hz), 6.02 (br s, 1H), 5.40 (br s, 1H), 5.35 (br s, 1H), 4.72 (m, 1H), 4.47 (m, 2H), 4.38 (m, 2H), 4.22 (t, 1H, J=5.7 Hz), 4.18 (m, 1H), 4.07 (m, 1H), 3.93 (m, 1H), 3.84 (m, 1H), 3.72 (m, 1H), 3.56 (m, 2H), 3.52 (m, 2H), 3.46 (m, 4H), 3.36 (m, 3H), 3.26 (m, 2H), 3.16 (m, 1H), 3.08 (m, 1H), 2.48 (m, 1H), 2.08 (m, 1H), 1.72 (m, 1H), 1.62 (m, 2H), 0.98 (d, 3H, J=6.1 Hz), 0.92 (d, 3H, J=6.6 Hz); ¹³C NMR (75 MHz, CD₃OD): δ 177.7, 173.4, 158.6, 145.2, 145.1, 142.6, 128.9, 128.3, 126.3 (triazole), 121.1, 111.7 (anomeric), 96.8 (anomeric), 96.2 (anomeric), 86.6, 81.5, 77.8, 76.5, 74.6, 73.9, 72.8, 72.1, 69.5, 69.3, 57.9, 55.9, 53.0, 51.4, 50.3, 43.0, 41.8, 40.3, 29.5, 29.1, 25.9, 23.6, 21.8; [α]_{D}²⁵=+32.0 (c 0.03, MeOH); EIMS: calcd. for C₄₉H₇₅N₁₂O₁₆⁺ 1087.53 Found: 1087.28 (M+H)⁺.

Compound 25. Yield=49%; R_{f} 0.15 (NH₄OH/MeOH/CH₂Cl₂, 2:5:7); ¹H NMR (300 MHz, CD₃OD): δ 7.99 (br s, 1H), 7.80 (d, 2H, J=7.3 Hz), 7.65 (d, 2H, J=7.6 Hz), 7.39 (t, 2H, J=7.3 Hz), 7.31 (t, 2H, J=7.3 Hz), 6.02 (br s, 1H), 5.43 (br s, 1H), 5.37 (br s, 1H), 4.72 (m, 1H), 4.52 (m, 2H), 4.44 (dd, 2H, J=4.5, 9.7 Hz), 4.37 (m, 6H), 4.20 (m, 2H), 4.13 (m, 2H), 3.90 (m, 2H), 3.74 (m, 1H), 3.64 (m, 1H), 3.49 (m, 6H), 3.28 (m, 2H), 3.16 (m, 1H), 3.08 (m, 2H), 2.48 (m, 1H), 2.08 (m, 1H), 1.72 (m, 2H), 1.56 (m, 4H), 0.97-0.91 (4s, 12H); ¹³C NMR (75 MHz, CD₃OD): δ 177.7, 173.4, 158.6, 145.2, 142.6, 128.9, 128.3, 126.4 (triazole), 121.1, 111.7 (anomeric), 96.8 (anomeric), 96.2 (anomeric), 86.4, 82.4, 81.2, 80.7, 80.3, 77.8, 76.6, 74.6, 73.8, 72.9, 72.1, 69.3, 69.2, 68.3, 58.0, 56.0, 55.0, 53.2, 51.4, 50.3, 42.8, 41.8, 40.3, 29.5, 28.9, 25.8, 23.6, 21.8; [α]_{D}²⁵=+28.0 (c 0.03, MeOH); EIMS: calcd. for C₅₅H₈₆N₁₃O₁₇⁺ 1200.34 Found: 1200.56 (M+H)⁺.

### Example 7

### Anti-Bacterial Experiments

The microbiological activities of the neomycin-peptide conjugates and kanamycin-peptide conjugates against both American Type Culture Collection (ATCC) reference and clinical strains of Staphylococcus aureus, MRSA, Staphylococcus epidermidis, MRSE, Enterococcus faecalis, Enterococcus faecium, Streptococcus pneumoniae, E. coli, Pseudomonas aeruginosa, Stenotrophomonas maltophilia, Acinetobacter baumannii, and Klebsiella pneumoniae were assessed. Special focus was given to assess activity against multi-drug resistant strains of these pathogens. Antibiotic susceptibility testing was performed using the macrobroth dilution method as per Clinical Laboratory Standards Institute-CLSI (formerly National Committee for Clinical and Laboratory Standards-NCCLS) (Clinical and Laboratory Standards Institute, 2006). MIC values defined as the lowest concentration of antimicrobial agent which inhibited the development of visible growth after 24 h at 37° C.

The antibacterial activities in the form of minimum inhibitory concentrations (MIC) in μg/mL of neomycin-peptide conjugates 12, 13, 21, 24 and 25 and kanamycin-peptide conjugates 17, 19 and 23 against Gram-positive, and Gram-negative organisms were determined and are shown in Table 3. These results demonstrate that neomycin B- and kanamycin A-peptide conjugates display potent antimicrobial activities against Gram-positive and Gram-negative organisms. For instance, conjugate 12 (a neomycin B- peptide conjugate) exhibits potent Gram-positive activities against S. aureus, methicillin resistant S. aureus (MRSA), S. epidermidis, and methicillin resistant S. epidermidis (MRSE) but also potent activity against Gram-negative E. coli (ATCC 25922). In addition, conjugates 13 and 17 exhibit potent activity against the Gram-negative bacilli K. pneumoniae and E. coli. Interestingly, a ≧16-fold enhancement in antibacterial activity against methicillin-resistant S. aureus (MRSA) is observed for conjugates 12 and 21 when compared to the parent aminoglycosides neomycin B and kanamycin A and a 8-fold enhancement is observed in kanamycin A-conjugate 19 when compared to kanamycin A against MRSE.

In general, neomycin B-peptide conjugates demonstrated greater activity against Gram-positive cocci while kanamycin A-peptide conjugates demonstrated greater activity against Gram-negative bacilli (Table 3). In addition, neomycin-peptide and kanamycin-peptide conjugates displayed similar activity versus both aminoglycoside susceptible and aminoglycoside resistant strains, suggesting that they display a different mechanism of action than aminoglycosides alone.

These results demonstrate that triazole aminoglycoside-(amino acid)ₙ conjugates exhibit potent antibacterial activity against Gram-positive and Gram-negative organisms. In particular, significantly enhanced activity against neomycin- and kanamycin-resistant strain of MRSA and kanamycin-resistant MRSE is observed. Moreover, triazole aminoglycoside-(amino acid)ₙ conjugates display similar activity versus both aminoglycoside antibiotic susceptible and aminoglycoside antibiotic resistant strains, suggesting that they display a different mechanism of action than aminoglycoside antibiotics alone.

TABLE 3
Representative minimal inhibitory concentrations (MIC) in μg/mL for
compounds 9, 12, 13, 17, 19, 21, 23, 24, 25 and neomycin B and kanamycin A
against various bacterial strains: ^{a}methicillin-resistant S. aureus (ATCC 33592);
^{ba}methicillin-resistant S. epidermidis.

| | Comp | | | | | | | | | | |
|---|---|---|---|---|---|---|---|---|---|---|---|
| strain | 9 | 12 | 13 | 17 | 19 | 21 | 23 | 24 | 25 | Neomycin B | Kanamycin A |
| S. aureus | 16 | 8 | 16 | 16 | 16 | 8 | 32 | 32 | 32 | 2 | 4 |
| ATCC 29213 | | | | | | | | | | | |
| MRSA ATCC | 64 | 16 | >256 | >256 | 32 | 16 | 32 | 32 | 32 | 256 | >512 |
| 33592^{a} | | | | | | | | | | | |
| S. epidermidis | 4 | 4 | 8 | 4 | 8 | 2 | 8 | 8 | 16 | 1 | 2 |
| ATCC 14990 | | | | | | | | | | | |
| MRSE CAN- | 8 | 8 | 8 | 128 | 16 | 4 | 16 | 16 | 16 | 0.5 | 128 |
| ICU 61589^{b} | | | | | | | | | | | |
| E. faecalis | n.d. | n.d. | 128 | 128 | n.d. | n.d. | n.d. | 128 | 128 | n.d. | n.d. |
| ATCC 29212 | | | | | | | | | | | |
| E. faecium | n.d. | | 128 | 256 | n.d. | n.d. | n.d. | 64 | 32 | n.d. | n.d. |
| ATCC 27270 | | | | | | | | | | | |
| S. pneumoniae | >512 | 64 | 128 | 64 | 64 | 64 | 64 | >256 | >128 | 64 | 8 |
| ATCC 49619 | | | | | | | | | | | |
| E. coli ATCC | 32 | 16 | 32 | 8 | 32 | 32 | 64 | 64 | 64 | 8 | 8 |
| 25922 | | | | | | | | | | | |
| E. coli ATCC | 32 | 32 | 32 | 128 | 32 | 64 | 64 | 64 | 128 | 4 | 16 |
| (Gent-R) | | | | | | | | | | | |
| CAN-ICU | | | | | | | | | | | |
| 61714 | | | | | | | | | | | |
| E. coli ATCC | 64 | n.d. | 256 | 32 | 32 | 64 | 64 | 64 | 128 | n.d. | 32 |
| (Amikacin | | | | | | | | | | | |
| 32) CAN-ICU | | | | | | | | | | | |
| 63074 | | | | | | | | | | | |
| P. aeruginosa | 512 | 128 | >256 | >256 | 128 | 128 | 128 | 256 | 128 | 512 | >512 |
| ATCC 27853 | | | | | | | | | | | |
| P. aeruginosa | 512 | 64 | 256 | >256 | 16 | 32 | 64 | 64 | 64 | 512 | >512 |
| (Gent-R) | | | | | | | | | | | |
| CAN-ICU | | | | | | | | | | | |
| 62308 | | | | | | | | | | | |
| S. maltophilia | n.d. | n.d. | >256 | >256 | n.d. | n.d. | n.d. | >256 | >256 | >512 | n.d. |
| CAN-ICU | | | | | | | | | | | |
| 62584 | | | | | | | | | | | |
| A. baumannii | n.d. | n.d. | >256 | 256 | n.d. | n.d. | n.d. | >256 | >256 | 64 | n.d. |
| CAN-ICU | | | | | | | | | | | |
| 63169 | | | | | | | | | | | |
| K. pneumoniae | n.d. | n.d. | 8 | 4 | n.d. | n.d. | n.d. | 128 | 256 | 1 | n.d. |
| ATCC 13883 | | | | | | | | | | | |

### Example 8

### Human Treatment With Triazole Aminoglycoside-(Amino Acid)ₙ Conjugates

This example describes an exemplary protocol to facilitate the treatment of a bacterial infection in a patient using a triazole aminoglycoside-(amino acid)ₙ conjugate. Patients may, but need not, have received previous anti-bacterial treatment.

A composition of the present invention is typically administered orally or topically in dosage unit formulations containing standard, well known non-toxic physiologically acceptable carriers, adjuvants, and/or vehicles as desired. The term “parenteral” as used herein includes subcutaneous injections, intravenous, intramuscular, intra-arterial injection, or infusion techniques. Triazole aminoglycoside-(amino acid)ₙ conjugates may be delivered to the patient before, after, or concurrently with any other anti-bacterial agent(s), if desired.

A typical treatment course comprises dosing over a 7-14 day period. Dosing may include 1-3 dosages per day (e.g., swallowing of a pill comprising a compound of the present invention three times a day). Upon election by the clinician, the regimen may be continued for days or weeks on a more frequent or less frequent basis (e.g., twice a day, four times a day, etc.) basis. Of course, these are only exemplary times for treatment, and the skilled practitioner will readily recognize that many other time-courses are possible.

To treat a bacterial infection using the methods and compositions described in the present invention, one will generally contact a target bacteria with a triazole aminoglycoside-(amino acid)ₙ conjugate. These compositions are provided in an amount effective to treat the infection, or, at a minimum, decrease side effects associated with the infection.

Regional delivery of a triazole aminoglycoside-(amino acid)ₙ conjugate is an efficient method for delivering a therapeutically effective dose to counteract the bacterial. Alternatively systemic delivery of a triazole aminoglycoside-(amino acid)ₙ conjugate may be appropriate. A therapeutic composition of the present invention may be administered to the patient directly at the site of the infection. This is in essence a topical treatment of the surface of the infection. The volume of the composition comprising the triazole aminoglycoside-(amino acid)ₙ conjugate should usually be sufficient to ensure that the infection is contacted by the triazole aminoglycoside-(amino acid)ₙ conjugate.

Clinical responses may be defined by acceptable measure. For example, a complete response may be defined by the disappearance of all measurable infection for at least a month. A partial response may be defined by a 50% or greater reduction of the number of excess white blood cells, wherein excess white blood cells is defined as an amount of white blood cells that exceeds a normal range.

Of course, the above-described treatment regimes may be altered in accordance with the knowledge gained from clinical trials, such as those described in Example 9. Those of skill in the art are able to take the information disclosed in this specification and optimize treatment regimes based on the results from the trials.

### Example 9

### Clinical Trials of the Use of Triazole Aminoglycoside-(Amino Acid)ₙ Conjugates in Treating Bacterial Infections

This example is concerned with the development of human treatment protocols using a triazole aminoglycoside-(amino acid)ₙ conjugate. These conjugates are of use in the clinical treatment of various bacterial infections in which infectious bacteria, such as multi-drug resistant infectious bacteria, play a role.

The various elements of conducting a clinical trial, including patient treatment and monitoring, are known to those of skill in the art in light of the present disclosure. The following information is being presented as a general guideline for studying triazole aminoglycoside-(amino acid)ₙ conjugates in clinical trials.

Patients with a bacterial infection, such as a bacterial infection of the abdomen, urinary tract, blood (bacteremia) or heart (endocarditis), are chosen for clinical study. Administration of a triazole aminoglycoside-(amino acid)ₙ conjugate may be orally or topically. The starting dose may be 5 mg/kg body weight. Three patients may be treated at each dose level. Dose escalation may be done by 100% increments (5 mg, 10 mg, 20 mg, 40 mg) until drug related toxicity is detected. Thereafter, dose escalation may proceed by 25% increments, if at all, depending on the tolerance of the patient.

The triazole aminoglycoside-(amino acid)ₙ conjugate may be administered over a 7 to 14 day period. The triazole aminoglycoside-(amino acid)ₙ conjugate may be administered alone or in combination with, for example, another anti-bacterial agent. The infusion given at any dose level is dependent upon the toxicity achieved after each. Increasing doses of the triazole aminoglycoside-(amino acid)ₙ conjugate in combination with an anti-bacterial agent is administered to groups of patients until approximately 60% of patients show unacceptable toxicity in any category. Doses that are ⅔ of this value could be defined as the safe dose.

Physical examination, visual assessment of the infection site and laboratory tests (e.g., white blood cell counts) should, of course, be performed before treatment and at intervals of about 3-4 weeks later. Laboratory studies should include CBC, differential and platelet count, urinalysis, SMA-12-100 (liver and renal function tests), and any other appropriate chemistry studies to determine the extent of the infection, or determine the cause of existing symptoms.

Clinical responses may be defined by acceptable measure. For example, a complete response may be defined by the disappearance of all measurable infection for at least a month. A partial response may be defined by a 50% or greater reduction of the number of excess white blood cells, wherein excess white blood cells is defined as an amount of white blood cells that exceeds a normal range.

All of the compositions and/or methods disclosed and claimed herein can be made and executed without undue experimentation in light of the present disclosure. While the compositions and methods of this invention have been described in terms of some embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### V. REFERENCES

The following references, to the extent that they provide exemplary procedural or other details supplementary to those set forth herein, are specifically incorporated herein by reference.
Alper et al., J. Am. Chem. Soc., 120:1965-1978, 1998.
Asensio et al., J. Am. Chem. Soc., 127:8278-8279, 2005.
Bastida et al., J. Am. Chem. Soc., 128:100-116, 2006.
Begg and Barclay, Br. J. Clin. Pharmacol., 39:597-603, 1995.
Bodansky, Peptide Chemistry, A Practical Textbook, 2^{nd} ed., Springer-Verlag, 1993.
Bunin, In: The Combinatorial Index, Academic Press, 1998.
Carter, et al., Nature, 407:340-348, 2000.
Cheshev et al., Org. Biomol. Chem., 4:3225-3227, 2006.
Clinical and Laboratory Standards Institute, M100-S16—CLSI/NCCLS M100-S15, 2006.
Czarnik and De Witt, In: A Practical Guide to Combinatorial Chemistry, American Chemical Society, 1-360, 1997.
Davies, Science, 264:375-382, 1994.
Davis, Microbiol. Rev., 51:341-350, 1987.
Deiters et al., J. Am. Chem. Soc., 725:11782-11783, 2003.
Disney and Barrett, Biochemistry, 46:11223-11230, 2007.
Evans et al., J. Amer. Chem. Soc., 112:4011-4030, 1990.
Fazio et al., J. Am. Chem. Soc. 724:14397-14402, 2002.
Fong and Berghuis, EMBO J., 21:2323-2331, 2002.
Girodeau et al., J. Antibiot., 37:150-158, 1984.
Gogoi et al., Nucleic Acids Res., 1-7, 2007.
Gordon et al., J. Med. Chem., 37:1233-1252; 1386-1401, 1994.
Grant (Ed.), Synthetic Peptides, A User's Guide, W. H. Freeman and Co., New York, 1992.
Greene and Wuts, Protecting Groups in Organic Synthesis, 2^{nd} Ed., Wiley, New York, 1991.
Greenberg et al., J. Am. Chem. Soc., 121:6527-6541, 1999.
Haddad et al., In: Glycochemistry Principles, Synthesis, and Application, Wang et al. (Eds.), Marcel Dekker, NY, 307-424, 2001.
Handbook of Pharmaceutical Salts: Properties, Selection and Use (Stahl & Wermuth, Eds.), Verlag Helvetica Chimica Acta, 2002.
Hanessian et al., J. Antibiot., 30:893-896, 1977.
Hanessian et al., Tetrahedron, 59:983-993, 2003.
Hoffmann et al., Helv. Chim. Acta, 85:265, 2002.
Hooper, In: Aminoglycoside Antibiotics, Springer-Verlag, NY, 1982.
Huisgen, Proc. Chem. Soc., 357-369, 1961.
Kolb and Sharpless, Drug Discovery Today, 8:1128-1137, 2003.
Kolb et al., Angew. Chem. Int. Ed., 40:2004-2021, 2001.
Lee et al., J. Am. Chem. Soc. 725:9588-9589, 2003.
Lewis et al., Angew. Chem. Int. Ed., 41:1053-1057, 2002.
Link and Tirrell, J. Am. Chem. Soc., 725:11164-11165, 2003.
Kumar et al., J. Med. Chem., 23:42-49, 1980.
Magnet and Blanchard, Chem. Rev., 105:477-497, 2005.
Meng et al., Angew. Chem. Int. Ed. 43:1255-1260, 2004.
Merrifield, Science, 232:341-347, 1986.
Michael et al., Bioorg. Med. Chem., 7:1361-1371, 1999.
Mingeot-Leclercq and Tulkens, Antimicrob. Agents Chemother., 43:1003-1012, 1999.
Mingeot-Leclercq et al., Antimicrob. Agent. Chemother., 43:727-737, 1999.
Papagianni, Biotechnol. Adv., 21:465, 2003.
Pu et al., J. Amer. Chem. Soc., 56:1280-1283, 1991.
Quader et al., J. Org. Chem., 72:1962-1979, 2007.
Remington's Pharmaceutical Sciences, 18th Ed., Mack Printing Company, 1990, Rodios, J. Heterocycl. Chem., 21:1169, 1984.
Rostovtsev et al., Angew. Chem., Int. Ed., 41:2596-2599, 2002.
Schoen and Kisfaludy, Synthesis, 303, 1986.
Shaw et al., Microbiol. Rev., 57:138-163, 1993.
Sitaram and Nagaraj, Curr. Pharm. Des., 8:727-742, 2002.
Smith and Baker, Curr. Drug Targets Infect. Disord., 2:143-160, 2002.
Smith and March (Eds.), March's Advanced Organic Chemistry: Reactions, Mechanisms, and Structure (March's Advanced Organic Chemistry), 2001.
Sonogashira et al., Tetrahedron Lett., 16:4467-4470, 1975.
Speers et al., J. Am. Chem. Soc., 725:4686-4687, 2003.
Sucheck et al., J. Am. Chem. Soc., 122:523-524, 2000.
Terret et al., Tetrahedron, 51:8135-8173, 1995.
Tornøe et al., J. Org. Chem., 67:3057, 2002.
Vakulenko and Mobashery, Clin. Microbiol. Rev., 16:430-450, 2003.
Wang et al., Org. Lett., 4:3997-4000, 2002.
Wang and Tor, Angew. Chem., Int. Ed. Engl., 32:109-111, 1993.
Wang et al., J. Am. Chem. Soc., 125:3192-3193, 2003.
Waskman et al., Proc. Staff Meet. Mayo Clin., 19:537-548, 1944.
Williams et al., J. Amer. Chem. Soc., 113:9276-9286, 1991.
Williams, In: Synthesis of Optically Active α-Amino Acids, Pergamon Press, 1989. Yoshikawa et al., Chem. Lett., 13:2097-2100, 1984.

1. A triazole aminoglycoside-(amino acid)ₙ conjugate, wherein at least one amino acid has been modified to comprise a triazolylmethyl linker that is bound to at least one aminoglycoside, and n=1-20.

2. The triazole aminoglycoside-(amino acid)ₙ conjugate of claim 1, wherein a side chain, an N-terminus, and/or a C-terminus of an amino acid has been modified to comprise a triazolylmethyl linker that is bound to at least one aminoglycoside.

3-4. (canceled)

5. The triazole aminoglycoside-(amino acid)ₙ conjugate of claim 1, wherein the aminoglycoside is bound to the triazolylmethyl linker at a primary hydroxy position, a secondary hydroxy position, a primary amino position, or a secondary amino position of the aminoglycoside.

6. The triazole aminoglycoside-(amino acid)ₙ conjugate of claim 5, wherein the aminoglycoside is bound to the triazolylmethyl linker at a primary hydroxy position of the aminoglycoside.

7. The triazole aminoglycoside-(amino acid)ₙ conjugate of claim 1, wherein the aminoglycoside is further defined as an aminoglycoside antibiotic.

8. The triazole aminoglycoside-(amino acid)ₙ conjugate of claim 7, wherein the aminoglycoside antibiotic is further defined as a neomycin, a kanamycin, amikacin, a gentamicin, neamine, a streptomycin, tobramycin, a hygromycin, or spectinomycin.

9-10. (canceled)

11. The triazole aminoglycoside-(amino acid)ₙ conjugate of claim 1, wherein n=1-20, and wherein each amino acid may be the same or different, and each amino acid is comprised in a single peptide.

12. (canceled)

13. The triazole aminoglycoside-(amino acid)ₙ conjugate of claim 12, wherein the single peptide comprises one or more amino acid residues selected from the group consisting of L- or D-glycyl, L- or D-alanyl, L- or D-valinyl, L- or D-leucyl, L- or D-isoleucyl, L- or D-threonyl, L- or D-seryl, L- or D-cysteinyl, L- or D-methionyl, L- or D-aspartyl, L- or D-glutamyl, L- or D-histidyl, L- or D-lysinyl, L- or D-asparagyl, L- or D-glutaminyl, L- or D-arginyl, L- or D-phenylalanyl, L- or D-tyrosyl, L- or D-tryptophyl, or L- or D-prolinyl.

14. (canceled)

15. The triazole aminoglycoside-(amino acid)ₙ conjugate of claim 11, wherein the single peptide is further defined as a cationic antimicrobial peptide.

16. The triazole aminoglycoside-(amino acid)ₙ conjugate of claim 11, wherein at least one amino acid of the single peptide further comprises a propargyl group.

17. The triazole aminoglycoside-(amino acid)ₙ conjugate of claim 16, wherein a side chain, an N-terminus, and/or a C-terminus of the amino acid of the single peptide has been modified to comprise a propargyl group.

18-20. (canceled)

21. The triazole aminoglycoside-(amino acid)ₙ conjugate of claim 11, wherein n=2 or 3 and the conjugate is further defined as 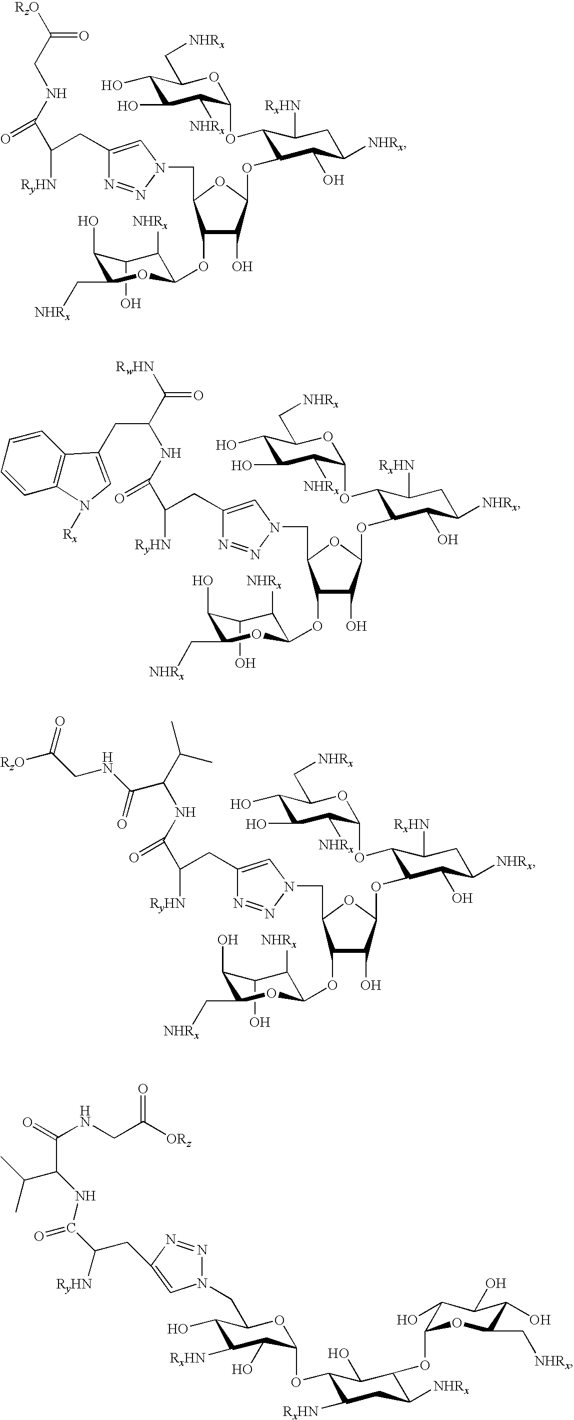 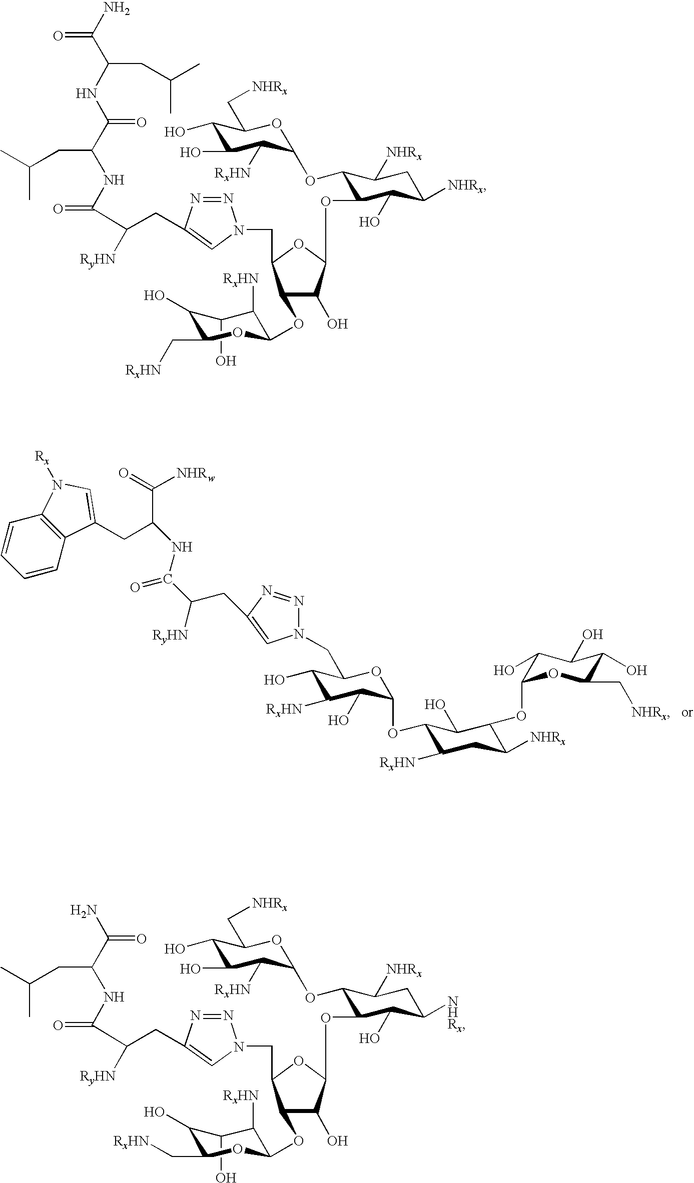
wherein:
R_{w}, Rₓ and R_{y} are each independently H or an amine protecting group; and
R_{z} is a carboxylic acid protecting group,
or salts thereof.

22. The triazole aminoglycoside-(amino acid)ₙ conjugate of claim 1, wherein at least two separate aminoglycosides are bound to at least two separate amino acids through two separate linkages that each comprise a triazolylmethyl linker.

23. The triazole aminoglycoside-(amino acid)ₙ conjugate of claim 22, further defined as 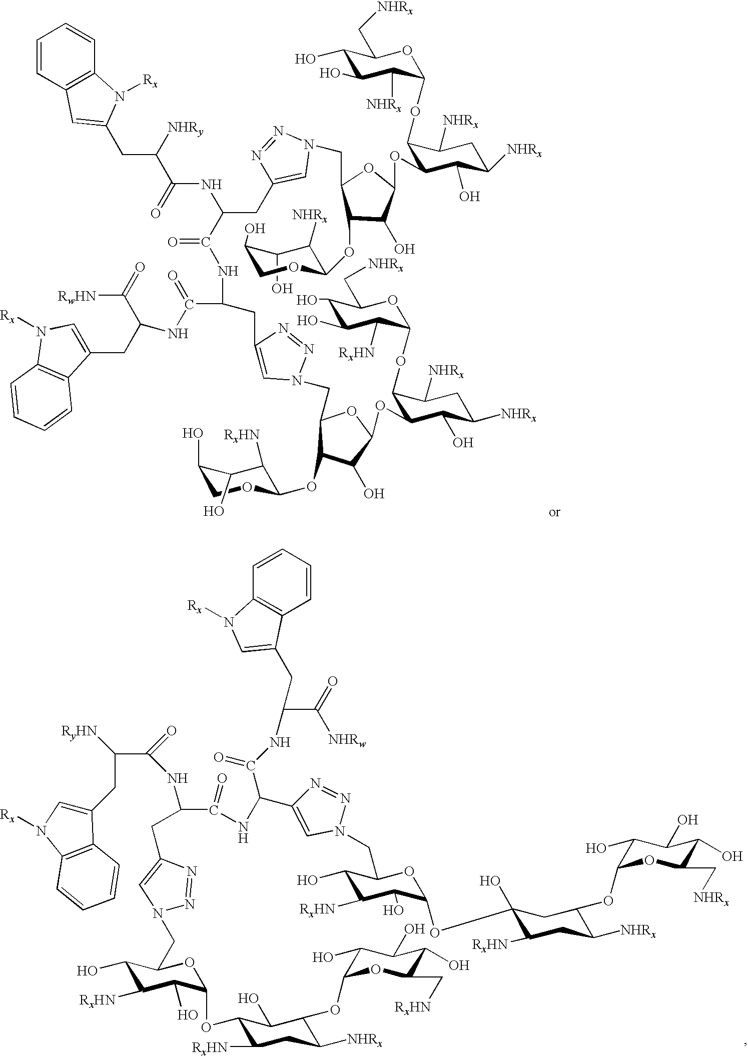
wherein:
Rₓ and R_{y} are each independently H or an amine protecting group; and
R_{z} is a carboxylic acid protecting group,
or salts thereof.

24. The triazole aminoglycoside-(amino acid)ₙ conjugate of claim 1, wherein the triazole aminoglycoside-(amino acid)ₙ conjugate is defined as a compound of formula (I): 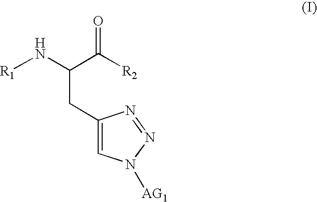
wherein:
R₁ is H, an amino protecting group, or (aa₁)ᵣ, wherein (aa₁) is an amino acid that is bound to the —NH— group of the compound of formula (I) through its carboxyl terminus such that an amide bond is formed, and r=1-19;
R₂ is —OR₃, wherein R₃ is H or a carboxylic acid protecting group, —NHR₄, wherein R₄ is H or an amino protecting group, or (aa₂)ₛ, wherein (aa₂) is an amino acid that is bound to the —C(O)— group of the compound of formula (I) such that an amide bond is formed, and s=1-19; and
AG₁ is an aminoglycoside, wherein the triazolyl is bound to AG₁ at a primary hydroxy position of AG₁,
wherein r+s≦20.

25. (canceled)

26. The triazole aminoglycoside-(amino acid)ₙ conjugate of claim 24, wherein R₁ is (aa₁)ᵣ, and r=1-19.

27. (canceled)

28. The triazole aminoglycoside-(amino acid)ₙ conjugate of claim 26, wherein the amino acid in the terminal position of (aa₁)ᵣ terminates in —NHR₅, wherein R₅ is H or an amino protecting group.

29. (canceled)

30. The triazole aminoglycoside-(amino acid)ₙ conjugate of claim 24, wherein R₂ is (aa₂), and s=1-19.

31. (canceled)

32. The triazole aminoglycoside-(amino acid)ₙ conjugate of claim 30, wherein the amino acid in the terminal position of (aa₂)ₛ terminates in —C(O)OR₆, wherein R₆ is —OH or a carboxylic acid protecting group, or —NHR₇, wherein R₇ is H or an amino protecting group.

33. The triazole aminoglycoside-(amino acid)ₙ conjugate of claim 24, wherein R₁ is (aa₁)ᵣ and R₂ is (aa₂)ₛ, and at least one amino acid of (aa₁)ᵣ or (aa₂)ₛ has to comprise a triazolylmethyl linker that is covalently bound to at least a second aminoglycoside (AG₂).

34. The triazole aminoglycoside-(amino acid)ₙ conjugate of claim 33, wherein the triazolylmethyl linker is bound to the second AG₂ at a primary hydroxy position of the AG₂.

35. The triazole aminoglycoside-(amino acid)ₙ conjugate of claim 24, wherein R₁ is (aa₁)ᵣ and R₂ is (aa₂)ₛ, and at least one amino acid of of (aa₁)ᵣ or (aa₂)ₛ comprises a propargyl moiety.

36. A peptide comprising the following moiety: 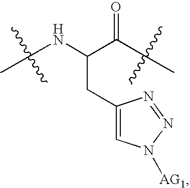
wherein AG₁ is an aminoglycoside that is bound to the triazolyl group at a primary hydroxy position of AG₁.

37. A pharmaceutical composition comprising a triazole aminoglycoside-(amino acid)ₙ conjugate, wherein the amino acid has been modified to comprise a triazolylmethyl linker that is bound to at least one aminoglycoside, and n=1-20, and a pharmaceutically acceptable carrier.

38. (canceled)

39. A method of making a triazole aminoglycoside-(amino acid)ₙ conjugate wherein n=1-20, comprising reacting a first azido-modified aminoglycoside with a propargyl-modified amino acid.

40. The method of claim 39, further comprising the step of obtaining an azido-modified aminoglycoside.

41. The method of claim 39, further comprising the step of obtaining a propargyl-modified amino acid.

42. The method of claim 39, wherein the azido-modified aminoglycoside is further defined as an aminoglycoside comprising a primary hydroxy position that has been modified to incorporate an azido group.

43. The method of claim 39, wherein the propargyl-modified amino acid is further defined as propargylglycine.

44. The method of claim 39, wherein n=2-20, and each amino acid may be the same or different and each amino acid is comprised in a single peptide.

45. The method of claim 44, wherein the single peptide further comprises a second amino acid comprising a propargyl group.

46. The method of claim 45, wherein the propargyl group of the second amino acid is reacted with a second azido-modified aminoglycoside, wherein the second aminoglycoside may be the same or different than the first aminoglycoside.

47. The method of claim 39, wherein the aminoglycoside-(amino acid)ₙ is further defined as an aminoglycoside antibiotic-(amino acid)ₙ and the azido-modified aminoglycoside is further defined as an azido-modified aminoglycoside antibiotic.

48. The method of claim 39, wherein the method is performed using solution phase peptide chemistry.

49. The method of claim 39, wherein the method is performed using solid phase peptide chemistry.

50. A method of making a compound of formula (I): 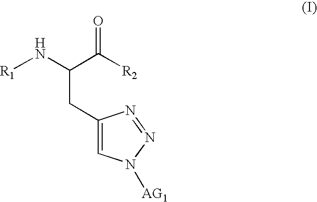
wherein:
R₁ is H, an amino protecting group, or (aa₁)ᵣ, wherein (aa₁) is an amino acid that is bound to the —NH— group of the compound of formula (I) through its carboxyl terminus such that an amide bond is formed, and r=1-19;
R₂ is —OR₃, wherein R₃ is H or a carboxylic acid protecting group, —NHR₄, wherein R₄ is H or an amino protecting group, or (aa₂)ₛ, wherein (aa₂) is an amino acid that is bound to the —C(O)— group of the compound of formula (I) such that an amide bond is formed, and s=1-19; and
AG₁ is an aminoglycoside, wherein the triazolyl is bound to AG₁ at a primary hydroxy position of AG₁,
wherein r=s <20;
comprising reacting an azido-modified-AG₁ with compound comprising propargylglycine.

51. The method of claim 50, wherein the compound comprising propargylglycine is further defined as a peptide comprising propargylglycine.

52. A method of treating a bacterial infection in a subject comprising administering to the subject an effective amount of a triazole aminoglycoside antibiotic-(amino acid)ₙ conjugate, wherein at least one amino acid has been modified to comprise a triazolylmethyl linker that is bound to at least one aminoglycoside, and n=1-20.

53. The method of claim 52, wherein the bacterial infection is caused by a multi-drug resistant bacteria.

54. The method of claim 52, wherein the bacteria is of any of the following types: Staphylococcus aureus, MRSA, Staphylococcus epidermidis, MRSE, Enterococcus faecalis, Enterococcus faecium, Streptococcus pneumoniae, E. coli, Pseudomonas aeruginosa, Stenotrophomonas maltophilia, Acinetobacter baumannii, Klebsiella pneumoniae or Mycobacterium tuberculosis.

55. The method of claim 52, wherein the minimum inhibitory concentration of the triazole aminoglycoside antibiotic-(amino acid)ₙ conjugate (MIC) is ≦150 μg/mL.

56. The method of claim 52, further comprising administration of a second antibacterial agent.

57. The method of claim 52, further comprising diagnosing the subject as needing treatment for the bacterial infection prior to administering the triazole aminoglycoside antibiotic-(amino acid)ₙ conjugate.

58. The method of claim 52, wherein the triazole aminoglycoside antibiotic-(amino acid)ₙ conjugate is topically administered to skin of the subject, wherein the skin has or is at risk of having a bacterial infection.

59-115. (canceled)
